SEQUENCE LISTING (2 sequences)

SEQ ID NO: 1 - DNA, 24 bp, Artificial
  forward primer
ctccagttcaggaacagtaaaccc

SEQ ID NO: 2 - DNA, 32 bp, Artificial
  reverse primer
ttgtgagctcagaaaggccttgtaagttggcg

(19) United States
(12) Patent Application Publication
(10) Pub. No.: US 2009/0105186 A1
(43) Pub. Date: Apr. 23, 2009

(54) Beta-L-N4-Hydroxycytosine Deoxynucleosides and their use as Pharmaceutical Agents in the Prophylaxis or Therapy of Viral Diseases

(75) Inventors: Eckart Matthes, Eggersdorf (DE); Martin von Janta-Lipinski, Berlin (DE); Hans Will, Hamburg (DE); Huseyin Sirma, Elmshorn (DE); Anneke Funk, Hamburg (DE)

Correspondence Address: JOYCE VON NATZMER;PEQUIGNOT + MYERS LLC, 200 Madison Avenue, Suite 1901, New York, NY, 10016, US

(73) Assignee: MAX-DELBRUECK-CENTRUM FUER MOLEKULARE MEDIZIN, Berlin (DE)

(21) Appl. No.: 11/577,677

(22) PCT Filed: Oct. 21, 2005
(86) PCT No.: PCT/EP2005/011555

§ 371 Date: Feb. 19, 2008

(30) Foreign Application Priority Data
Oct. 21, 2004 (DE) 102004051804.1

(51) Int. Cl. A61K 31/7068; C07H 19/067; A61P 35/00; A61P 31/18

(52) U.S. Cl. 514/49; 536/28.5

(57) ABSTRACT

The invention relates to β-L-N4-hydroxycytosine nucleo-sides, pharmaceutical agents comprising same, and to the use of said β-L-N4-hydroxycytosine nucleosides and pharmaceutical agents in the prophylaxis or therapy of an infection caused by hepatitis B virus (HBV) or human immunodeficiency virus (HIV). The invention also relates to a method for the preparation of said β-L-nucleoside analogs.

The invention relates to novel β-L-N4-hydroxycytosine nucleosides of general formula I

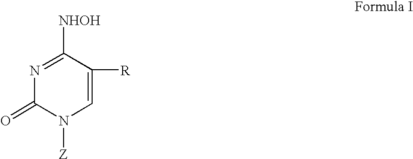

wherein:
R═H, halogen (F, Cl, Br, I), C₁-C₃ alkyl, and

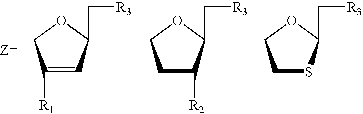

wherein

### R₁═H, F;

### R₂═H, F, OH, N₃; and

R₃═OH, O-acetyl, O-palmitoyl, alkoxycarbonyl, carbamate, phosphonate, monophosphate, bis(S-acyl-2-thioethyl) phosphate, diphosphate or triphosphate, and their use as pharmaceutical active substances or agents in the prophylaxis and/or treatment of infections caused in particular by hepatitis B virus (HBV) and human immunodeficiency virus (HIV).

The β-L-N4-hydroxycytosine nucleosides and the acceptable salts or prodrugs thereof can be used alone or in combination with other β-L-nucleosides, with 3-deazauridine or with other anti-HBV-effective compounds. Fields of use of the invention are medicine and the pharmaceutical industry.

### RELATED ART

HBV is the agent that triggers hepatitis B—an infectious disease, the chronic course of which affects about 350 million people worldwide, and particularly in Southeast Asia, Africa and South America. In a large number of cases, hepatitis B virus infections lead to eventual death as a result of liver function failure. Moreover, the chronic course is associated with a massively increased risk of primary liver carcinoma which, in China alone, results in about one million new cases of disease each year.

While the precise mechanism through which HBV can induce liver tumors remains unknown, it must be assumed that tumor induction is closely associated with HBV-induced chronic inflammation, developing cirrhosis and regeneration processes of the liver tissue.

The vaccine produced by genetic engineering, which has been available for many years, is not suitable for the treatment of hepatitis B virus infections because it fails to help persons already infected and is unable to stop the chronic course mentioned above.

In recent years, α-interferon produced by genetic engineering, in particular, has been found useful in the treatment of HBV infections. It is a cytokin with broad antiviral and immunomodulating activity. However, it is effective in only about 33% of the patients, entails considerable side effects, and cannot be administered on the oral route.

One nucleoside derivative applied with success and approved by the US Food and Drug Administration, as well as in Germany, is lamivudine (β-L-2′,3′-dideoxy-3′-thiacytidine), also known as thiacytidine (3TC), which has been described by Liotta et al. in U.S. Pat. No. 5,539,116. It is remarkable for its high efficacy both in HbeAg-positive and HbeAg-negative patients and has scarcely any side effects.

Although rapid decline of HBV DNA and normalization of the alanine transferase activity in serum is found in such treatment, HBV apparently cannot be completely eliminated from the liver under such therapy, so that re-onset of a hepatitis B virus infection is possible in many cases even after completion of a one-year treatment. Attempts are being made to prevent the above course by extending the treatment to several years, in the hope that HBV could be eliminated completely (Alberti et al., J Med Virol 2002, 67: 458-462).

However, such therapies are associated with an increasing risk of resistance to lamivudine, which can be as high as 45-55% after the second year of treatment (Liaw et al., Gastroenterology 2000, 119: 172-180).

The development of additional effective compounds is therefore an urgent necessity in order to replace the monotherapy by a combination therapy which not only can be more effective but can also substantially reduce the risk of resistance, as has been found in long-term treatment of HIV infections (Richman, Nature 2001, 410: 995-1000; Yeni et al., JAMA 2004, 292: 251-265).

Lamivudine belongs to a group of so-called β-L-nucleosides. They are enantiomeric compounds of naturally occurring β-D-nucleosides and, far a long time, have been regarded as defying enzymatic metabolization and therefore as inactive in biological systems.

This dogma was relativized for the first time in 1992 by the findings of Spadari et al. who had discovered that β-L-thymidine, while not being reacted by cellular thymidine kinase 1, is a substrate of the corresponding enzyme of herpes simplex virus 1 (Spadari et al., J Med Chem 1992, 35: 4214-4220). It has later been found that β-L-nucleosides can be substrates or inhibitors not only to some viral, but also to some cellular enzymes (Review: Maury, Antiviral Chem Chemother 2000, 11: 165-190).

In the following years, a variety of β-L-nucleoside analogs have been synthesized in pure form, among which—in addition to the above-mentioned lamivudine (3TC; β-L-2′,3′-dideoxy-3′-thiacytidine; Jeong et al., J Med Chem 1993, 36: 181-195)-emtricitabine (L-FTC; β-L-2′,3′-dideoxy-5-fluoro-3′-thiacytidine; Furman et al., Antimicrob Agents & Chemother 1992, 36: 2686-2692), β-L-2′-fluoro-5-methylarabino-furanosyluracil (L-FMAU; clevudine; Chu et al., Antimicrob Agents & Chemother 1995, 39: 979-981), β-L-2′,3′-dideoxycytidine and β-L-2′,3′-dideoxy-5-fluorocytidine (L-ddC, L-ddFC; Lin et al., J Med Chem 1994, 37: 798-803), β-L-2′,3′-dideoxy-2′,3′-didehydrocytidine and β-L-2′,3′-dideoxy-2′,3′-didehydro-5-fluorocytidine (L-d4C and L-d4FC; Lin et al., J Med Chem 1996, 39: 1757-1759), and β-L-thymidine (L-TdR; telbivudine; by Janta Lipinski et al., J Med. Chem. 1998, 41: 2040-2046; Bryant et al., Antimicrob Agents & Chemother 2001, 45: 229-235) have been found to be the most effective and promising inhibitors of HBV replication in vitro and in vivo, which are remarkable for their—in some cases—extremely low cytotoxicity. Among the D-nucleosides, entecavir (BMS 200475), a carbocyclic deoxyguanosine derivative (Innaimo et al., Antimicrob Agents & Chemother 1997, 41: 1444-1448), should be mentioned in particular, which has proven to be superior to lamivudine in the treatment of hepatitis B infections in an initial clinical study (Lai et al., Gastroenterology 2002, 123: 1831-1838).

Another promising purine nucleoside of the D series is 2′,3′-dideoxy-3′-fluoroguanosine (Matthes et al., Antimicrob Agents & Chemother 1991, 1254-1257; Hafkemeyer et al., Antimicrob Agents & Chemother 1996, 40: 792-794; Löfgren et al., J Viral Hepat 1996, 3: 61-65).

Further syntheses of L-nucleosides have been described in Mugnaini et al., Bioorg Med Chem 2003, 11: 357-366; Marquez et al., J Med Chem 1990, 33: 978; Lee et al., Nucleosides & Nucleotides 1999, 18: 537-540; Faraj et al., Nucleosides & Nucleotides 1997, 16: 1287-1290; Song et al., J Med Chem 2001, 44: 3985-3993; Kotra et al., J Med Chem 1997, 40: 1944; Choi et al., Organic Lett 2002, 4: 305-307; Gumina et al., Curr Top Med Chem 2002, 2: 1065-1086; Holy, Tetrahedron Lett. 1971, 189-193; Holy, Collect Czech Chem Commun 1972, 37: 4072-4082; and, in addition, the following patents describe β-L-nucleosides as potential virustatic agents: Gosselin et al., U.S. Pat. No. 6,395,716, Schinazi et al., US 2002-0107221 A1; Chu et al., U.S. Pat. No. 5,565,438, U.S. Pat. No. 5,567,688, U.S. Pat. No. 5,587,362, WO 92/18517 of the Yale University and University of Georgia Research Foundation, Inc.

In addition to β-L-cytosine nucleosides with non-modified cytosine as in β-L-deoxycytidine (Bryant et al., Antimicrob Agents & Chemother 2001, 45: 229-235), β-L-2′,3′-dideoxycytidine (L-ddC; Lin et al., J Med Chem 1994, 37: 798-803), β-L-2′,3′-dideoxy-2′,3′-didehydrocytidine (L-d4C; Lin et al., J Med Chem 1996, 39: 1757-1759), β-L-2′-fluoroarabinofuranosylcytosine (L-FAC; Ma et al., J Med Chem 1996, 39: 2835-2843), β-L-arabinofuranosylcytosine (L-AraC; Chu et al., U.S. Pat. No. 5,567,688), β-L-2′,3′-dideoxy-2′,3′-didehydro-2′-fluorocytidine (L-2′FddeC; Lee et al., J Med Chem 1999, 42: 1320-1328), some 5-modified cytosine derivatives have also been synthesized and investigated, especially 5-fluorocytosine derivatives which are either more effective than compounds with non-modified bases, such as β-L-2′,3′-dideoxy-2′,3′-didehydro-5-fluorocytidine (L-d4FC; Lin et al., J Med Chem 1996, 39: 1757-1759), equally effective, such as β-L-2′,3′-dideoxy-5-fluorocytidine (L-ddFC; Lin et al., J Med Chem 1994, 37: 798-803) or β-L-2′,3′-dideoxy-2′,3′-didehydro-2′-fluoro-5-fluorocytidine (L-2′F-ddeFC; Lee et al., J Med Chem 1999, 42: 1320-1328), less effective than β-L-2′-deoxy-5-fluorocytidine (L-FdC; Bryant et al., Antimicrob Agents & Chemother 2001, 45: 229-235), or exhibit no effect with respect to HBV replication, such as β-L-2′-fluoroarabinofuranosyl-5-fluorocytosine (L-FAFC; Ma et al., J Med Chem 1996, 39: 2835-2843) or β-L-arabinofuranosyl-5-fluorocytosine (L-AraFC; Griffon et al., Eur J Med Chem 2001, 36: 447-460).

Likewise, the following 5-chloro-, bromo- and methyl-modified L-cytosine nucleosides have been described as ineffective or sparingly effective: β-L-deoxy-5-chlorocyticine (CldC; Bryant et al., Antimicrob Agents & Chemother 2001, 45: 229-235), β-L-2′-fluoroarabinofuranosyl-5-chlorocytidine, β-L-2′-fluoroarabinofuranosyl-5-bromocytosine (L-FAC1C, L-FABrC; Ma et al., J Med Chem 1996, 39: 2835-2843), β-L-2′,3′-dideoxy-3′-thia-5-methylcytidine, β-L-2′,3′-dideoxy-3′-thia-5-bromocytidine, β-L-2′,3′-dideoxy-3′-thia-5-chlorocytidine and β-L-2′,3′-dideoxy-3′-fluoro-5-methylcytidine (Dong et al., Proc Natl Acad Sci USA 1991, 88: 8495-8499; Matthes et al., unpublished) and, in addition, some β-L-5-methylcytosine nucleosides have been described as effective to HBV infections (Matthes et al. PCT patent application PCT/DE2004/002051).

Some of the above-mentioned L-nucleosides are not only effective inhibitors of HBV replication, but also of HIV replication. Thus, for example, lamivudine has also been approved for the treatment of HIV infections. Other β-L-cytosine nucleosides already mentioned above, such as L-ddC, L-d4C, L-d4FC, and FTC, are also strong inhibitors of HIV replication, whose importance for therapy is to have new effective compounds available for combination therapy, thus providing the capability of coping with development of resistance (Menendez-Arias, Trends Pharmacol Sci 2002, 23: 381-388).

In addition, there are a number of β-L-nucleosides inhibiting HBV replication only (e.g. L-FMAU, L-TdR, L-CdR, L-3′FddC, L-d4C) and others inhibiting HIV replication only (e.g. abacavir).

All of the above-mentioned β-L-nucleosides are incorporated by HBV- or HIV-infected cells and must be converted into the nucleoside triphosphates by cellular enzymes. As a rule, this takes place in a step-by-step fashion. Instead of the nucleosides, however, it is also possible to use suitable nucleoside monophosphate triesters wherein the two negative phosphate charges are masked by ester bonds, allowing incorporation of said nucleoside monophosphate triesters in cells. Esterases in the cell liberate the nucleoside monophosphate therefrom, so that the first necessary and sometimes absent phosphorylation step of the nucleoside is circumvented in the cell in this way. Phosphoric diesters, e.g. linked with S-acyl-2-thioethyl groups (SATE), were found to be suitable nucleoside monophosphate prodrugs (Lefebvre et al., J Med Chem 1995, 38: 3941-3950; Peyrottes et al., Mini Rev Med Chem 2004, 4: 395-408).

It is only in the form of triphosphates where the nucleosides can bind their actual target, i.e. the HBV DNA polymerase or reverse transcriptase, in competition with normal substrates and give strong inhibition. As a consequence, the viral genomes can no longer by synthesized, and virus production comes to a standstill. Such inhibition must be selective, i.e., must be restricted to the viral polymerases and must not co-involve the cellular DNA polymerases, because otherwise—as a consequence of inhibition of the synthesis of cellular DNA—growth of rapidly proliferating cells would be impaired.

The invention is based on the object of developing new, antivirally effective β-L-N4-hydroxycytosine nucleosides effective against hepatitis B virus infections and HIV infections and exhibiting high efficacy against said infections, while having good tolerability and low toxicity.

Surprisingly, new β-L-N4-hydroxycytosine deoxynucleoside derivatives according to general formula I

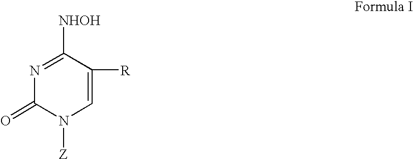

wherein:
R═H, halogen (F, Cl, Br, I), C₁-C₃ alkyl, and

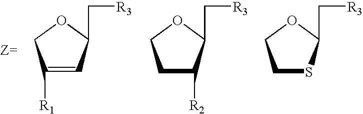

wherein

### R₁H, F;

### R₂H, F, OH, N₃; and

R₃═OH, O-acetyl, O-palmitoyl, alkoxycarbonyl, carbamate, phosphonate, monophosphate, bis(S-acyl-2-thioethyl) phosphate, diphosphate or triphosphate,
exhibit high antiviral activity against HBV and HIV.

Preferred are β-L-nucleosides in accordance with general formula I, wherein

R═H, F, Cl, Br, I or CH₃, and Z and R₁, R₂ and R₃ have the above-mentioned meanings.

Particularly preferred are β-L-nucleosides in accordance with general formula I, wherein

R═H, F or CH₃, and Z has the above-mentioned meanings, and
R₁═H or F, preferably H,

### R₂═H, F, OH or N₃, and

### R₃═OH.

The following were found to be particularly effective:
β-L-N4-hydroxydeoxycytidine (L-HyCdR),
β-L-5-methyl-N4-hydroxydeoxycytidine (L-HyMetCdR),
β-L-5-fluoro-N4-hydroxydeoxycytidine (L-HyFCdR),
β-L-2′ 3′-dideoxy-N4-hydroxycytidine (L-HyddC),
β-L-2′, 3′-dideoxy-5-fluoro-N4-hydroxycytidine L-HyddFC)
β-L-2′,3′-didehydro-2′, 3′-dideoxy-N4-hydroxycytidine (L-HyddeC),
β-L-2′,3′-didehydro-2′,3′-dideoxy-5-fluoro-N4-hydroxycytidine (L-HyddeFC),
β-L-2′ 3′-didehydro-2′ 3′-dideoxy-5-methyl-N4-hydroxycytidine (L-ddeMetC),
β-L-2′,3′-didehydro-2′,3′-dideoxy-2′-fluoro-N4-hydroxycytidine (L-HyFddeC),
β-L-2′,3′-dideoxy-3′-thia-N4-hydroxycytidine (Hy3TC),
β-L-2′ 3′-dideoxy-3′-thia-5-fluoro-N4-hydroxycytidine (HyFTC),
β-L-3′-azido-2′,3′-dideoxy-N4-hydroxycytidine (L-N₃HyCdR),
β-L-3′-azido-2′,3′-dideoxy-5-fluoro-N4-hydroxycytidine (L-N₃HyFCdR),
β-L-3′-azido-2′,3′-dideoxy-5-methyl-N4-hydroxycytidine,
β-L-3′-fluoro-2′,3′-dideoxy-N4-hydroxycytidine (L-FHyCdR).

In the β-D series, N4-hydroxydeoxycytidine has been known for many years. However, its rapid cleavage into cytosine and uracil has prevented in vivo utilization of its effects on cell proliferation (Nelson et al., Mol Pharmacol of 1966, 2: 248-258). Strong inhibition of thymidylate synthase has been described as cause of the antiproliferative effects (Goldstein et al., J Med Chem 1984, 27: 1259-1262), and this has led to the synthesis of other derivatives of β-D-N4-hydroxydeoxycytidine, namely, 5-halogen- and 5-hydroxymethyl-modified analogs which are also inhibitors of thymidylate synthase (Rode et al., Biochemistry 1990, 29: 10835-10842; Felczak et al., J Med Chem 2000, 43: 4647-4656). β-D-5-methyl-N4-hydroxydeoxycytidine and, in particular, the ribonucleoside β-D-N4-hydroxycytidine haze become known through their mutagenic effect in bacteria (Janion, Mut Res 1978, 56: 225-234; Sledziewska et al., Mut Res 1980, 70: 11-16).

More recently, said ribonucleoside, i.e., β-D-N4-hydroxycytidine, was found to be a strong inhibitor of the replication of hepatitis C virus (HCV) and bovine viral diarrhoea virus (BVDV) (Stuyver et al., Antimicrob Agents Chemother 2003, 47: 244-254), and this has induced further chemical modifications. Thus, β-D-3′-deoxy-N4-hydroxycytidine has been prepared and, in addition, the 5 position of the pyrimidine ring has been modified by halogen, methyl or 5-trifluoromethyl groups. Moreover, the synthesis of the corresponding enantiomeric 5-modified β-L-3′-deoxy-N4-hydroxycytidine derivatives has been described in the same paper for the first time, and all of the above derivatives were found to be ineffective to HVC (Hollecker et al., Antiviral Chem Chemother 2004, 14: 33-55).

On the other hand, β-L-N4-hydroxycytosine nucleosides as claimed herein are as yet unknown.

More specifically, the invention is therefore directed to the new β-L-N4-hydroxycytosine nucleosides of general formula I, to their application in the production of pharmaceutical agents, to pharmaceutical agents including these compounds, and to pharmaceutical agents including said compounds in combination with other pharmaceuticals, particularly in combination preparations with 3-deazauridine. Simultaneous application e.g. with 3-deazauridine significantly increases the efficacy.

3-Deazauridine activates the cellular deoxycytidine kinase and, in addition, the triphosphate thereof, formed intracellularly, is capable of inhibiting the cellular CTP synthase (Gao et al., Nucleosides Nucleotides Nucleic Acids 2000, 19: 371-377). As a consequence of the above two effects on the cellular deoxycytidine metabolism, 3-deazauridine gives rise to increased triphosphate levels of the β-L-N4-hydroxycytosine nucleosides of the invention, thereby massively increasing their efficacy with respect to HBV and HIV replication.

Surprisingly, it was found that the nucleosides according to the invention, i.e., the β-L-Hydroxycytosine nucleosides, can be used with high antiviral activity against selected viruses, especially against hepatitis viruses, preferably against hepatitis B virus.

In a preferred embodiment of the invention, derivatives of the inventive nucleosides are used. This may concern structures having modifications which, in particular, increase the antiviral activity. However, this may also concern a salt, a phosphonate, a monophosphate, a diphosphate, a triphosphate, an ester or a salt of such ester. Advantageously, such compounds can be used effectively in antiviral prophylaxis and therapy and exhibit only minor or no side effects at all.

The preparation of the compounds according to the invention is effected by means of per se known procedures, using modification of β-L-uridine or β-L-thymidine or condensation of modified β-L-sugars with a heterocycle such as 5-fluorouracil (Horwitz et al., J Org Chem 1967, 32: 817-818; Martin et al., J Med Chem 1990, 33: 2137-2145; Warshaw et al., J Med Chem 1990, 33: 1663-1666).

It is possible, for example, that the nucleosides in combination with other therapeutic, preferably antiviral agents have a synergistic effect by increasing the therapeutic effect in an additive or non-additive fashion, particularly by increasing the therapeutic index and/or reducing the risk of toxicity inherent in each single compound. Accordingly, the nucleosides of the invention preferably can also be used in combination therapies, including a wide variety of combinations with well-known therapeutic agents and pharmaceutically acceptable carriers. Of course, veterinary uses are also possible, as well as feed additives for all vertebrates. Particularly preferred is the use in humans. According to the explications above, the nucleosides of the invention can be used as drugs in a particularly preferred fashion. To this end, the nucleosides can be used alone, as a salt or derivative or as a composition. Pharmaceutically tolerable salts of compounds of the present invention include those derived from pharmaceutically tolerable inorganic and organic acids and bases. Examples of suitable acids include hydrochloric, hydrobromic, sulfuric, nitric, perchloric, fumaric, maleic, phosphoric, glycolic, lactic, salicylic, succinic, p-toluenesulfonic, tartaric, acetic, citric, methanesulfonic, ethanesulfonic, formic, benzoic, malonic, naphthalene-2-sulfonic and benzenesulfonic acids. Preferred acids include hydrochloric, sulfuric, methanesulfonic and ethanesulfonic acids. Most preferred is methanesulfonic acid. Other acids, such as oxalic acid, although not being pharmaceutically tolerable themselves, can be used in the production of salts usable as intermediate products in obtaining the compounds of the invention and their pharmaceutically tolerable acid addition salts.

Salts derived from suitable bases include alkali metal (e.g. sodium), alkaline earth metal (e.g. magnesium), ammonium and N(C₁₋₄ alkyl)₄⁺ salts.

Combinations of substituents and variables presented by this invention are preferably those resulting in the formation of stable compounds. The term “stable” as used herein relates to compounds having sufficient stability to allow preparation and maintain the integrity of the compound for a period of time sufficient to allow the use thereof for the purposes described in detail herein (for example, therapeutic or prophylactic administration to a mammal or use in affinity-chromatographic applications). Typically, such compounds are stable for at least one week at a temperature of 40° C. or less and in absence of moisture or other chemically reactive conditions.

The compounds of the present invention can be used in the form of salts derived from inorganic or organic acids. For example, such acid salts include the following: acetate, adipate, alginate, aspartate, benzoate, benzenesulfonate, bisulfate, citrate, camphorate, camphersulfonate, cyclopentanepropionate, digluconate, dodecylsulfate, ethanesulfonate, fumarate, glucoheptanoate, glycerophosphate, hemisulfate, heptanoate, hexanoate, hydrochloride, hydrobromide, hydroiodide, 2-hydroxyethanesulfonate, lactate, maleate, methanesulfonate, 2-naphthalenesulfonate, nicotinate, oxalate, palmoate, pectinate, persulfate, 3-phenylpropionate, picrate, pivalate, propionate, succinate, tartrate, thiocyanate, tosylate and undecanoate.

The invention also relates to nucleic acids or oligonucleotide containing as building blocks one or more nucleosides of the invention. Such nucleic acids can be produced according to methods well-known to those skilled in the art, and in a preferred fashion the nucleic acids of the invention are constituted of from 2 to 5000, preferably from 10 to 100 nucleoside building blocks, more preferably from 20 to 40 nucleoside building blocks. The nucleic acids or oligonucleotides of the invention containing central deoxycytidyl-deoxyguanosine (CpG) dinucleotides which were shown to possess immunostimulatory effects. The invention includes immunostimulatory effects of nucleic acids or oligonucleotides in which the deoxycytidine of the CpG motif is replaced by β-L-N4-hydroxydeoxycytidine or β-L-N4-hydroxy-5-fluorodeoxycytidine or β-L-N4-hydroxy-5-methyldeoxycytidine. These nucleic acids or oliogonucleotide can be preferably used for the treatment of cancer, HBV- and HIV-infections, asthma and allergic diseases.

The synthetic nucleic acids or antisense nucleic acids according to the invention can be present in the form of a therapeutic composition or formulation which can be used to stimulate the immunosystem in cancer patients, to treat human hepatitis-infections asthma or allergic diseases. They can be used as part of a pharmaceutical composition in combination with a physiologically and/or pharmaceutically tolerable carrier. The properties of the carrier will depend on the route of administration. In addition to synthetic nucleic acid and carrier, such a composition may include diluents, fillers, salts, buffers, stabilizers, solvents and other well-known materials. The pharmaceutical composition of the invention may also include other active factors and/or substances enhancing the inhibition of HBV expression. Furthermore, the pharmaceutical composition of the invention may include other chemotherapeutical agents for the treatment of liver carcinomas. Such additional factors and/or substances can be incorporated in the pharmaceutical composition in order to create a synergistic effect together with the synthetic nucleic acids of the invention or reduce side effects of the synthetic nucleic acids according to the invention. On the other hand, the synthetic nucleic acids of the invention can be incorporated in formulations of a particular anti-HBV or anti-cancer factor and/or substance to reduce the side effects of said anti-HBV factor and/or substance.

The pharmaceutical composition of the invention can be pre-sent in the form of a liposome wherein the synthetic nucleic acids of the invention, in addition to other pharmaceutically tolerable carriers, are combined with amphipathic substances such as lipids, which are present as micelles in one form of aggregation, insoluble monolayers, liquid crystals or lamellar layers present in an aqueous solution. Suitable lipids for a liposomal formulation include—but are not limited to—monoglycerides, diclycerides, sulfatides, lysolecithin, phospholipids, saponins, bile acids and the like. The preparation of such Liposomal formulations proceeds in a per se known manner and is well-known to those skilled in the art. Furthermore, the pharmaceutical composition of the invention may include other lipid carriers such as lipofectamine or cyclodextrins and the like, thereby enhancing the supply of said nucleic acids to the cells, or it may include polymers with delayed release.

The invention also relates to a pharmaceutical agent comprising at least one nucleoside and/or nucleic acid according to the invention, optionally together with conventional auxiliaries, preferably carriers, adjuvants and/or vehicles. A pharmaceutical agent in the meaning of the invention is any agent in the field of medicine, which can be used in the prophylaxis, diagnosis, therapy, follow-up or aftercare of patients who have come in contact with viruses, including hepatitis viruses, in such a way that a pathogenic modification of the overall condition or of the condition of particular parts of the organism could establish at least temporarily. Thus, for example, the pharmaceutical agent in the meaning of the invention can be a vaccine, an immunotherapeutic or immunoprophylactic agent. The pharmaceutical agent in the meaning of the invention may comprise the nucleosides or nucleic acids of the invention and/or an acceptable salt or components thereof. For example, salts of inorganic acids may be concerned, such as phosphoric acid, or salts of organic acids. Furthermore, the salts can be free of carboxyl groups and derived from inorganic bases, such as sodium, potassium, ammonium, calcium or iron hydroxides, or from organic bases such as isopropylamine, trimethylamine, 2-ethylaminoethanol, histidine and others. Examples of liquid carriers are sterile aqueous solutions including no additional materials or active ingredients, such as water, or those including a buffer such as sodium phosphate with a physiological pH value or a physiological salt solution or both, e.g. phosphate-buffered sodium chloride solution. Other liquid carriers may comprise more than just one buffer salt, e.g. sodium and potassium chloride, dextrose, propylene glycol, polyethylene glycol or others.

Liquid compositions of said pharmaceutical agents may additionally comprise a liquid phase, also one excluding water. Examples of such additional liquid phases are glycerol, vegetable oils, organic esters or water-oil emulsions. The pharmaceutical composition or pharmaceutical agent typically includes a content of at least 0.1 wt.-% of nucleosides or nucleic acids of the invention, relative to the overall pharmaceutical composition. The respective dose or dose range for administering the pharmaceutical agent of the invention method is in an amount sufficient to achieve the desired prophylactic or therapeutic antiviral effect. The dose should not be selected in such a way that indesirable side effects would dominate. In general, the dose will vary with the age, constitution, sex of a patient, and obviously with respect to the severity of the disease. The individual dose can be adjusted both with respect to the primary disease and with respect to ensuing additional complications. The exact dose can be detected by a person skilled in the art, using well-known means and methods, e.g. by determining the virus titer as a function of the dose or as a function of the vaccination scheme or of the pharmaceutical carriers and the like. Depending on the patient, the dose can be selected individually. For example, a dose of pharmaceutical agent tolerated by a patient can be one where the local level in plasma or in individual organs ranges from 0.1 to 10,000 μM, preferably between 1 and 100 μM. Alternatively, the dose can also be estimated relative to the body weight of the patient. In this event, for example, a typical dose of pharmaceutical agent would be adjusted in a range between 0.1 μg to 100 μg per kg body weight, preferably between 1 and 50 μg/kg. Furthermore, it is also possible to determine the dose with respect to individual organs rather than the overall patient. For example, this would apply to those cases where the pharmaceutical agent of the invention, incorporated in the respective patient e.g. in a biopolymer, is placed near particular organs by means of surgery. A number of biopolymers capable of liberating the nucleosides or nucleic acids in a desired manner are well-known to those skilled in the art. For example, such a gel may include from 1 to 1000 μg of compounds or pharmaceutical agent of the invention per ml gel composition, preferably between 5 and 500 μg/ml, and more preferably between 10 and 100 mg/ml. In this event, the therapeutic agent will be administered in the form of a solid, gel-like or liquid composition.

In a preferred fashion the pharmaceutical agent may also include one or more additional agents from the group of antiviral, fungicidal or antibacterial agents and/or immunostimulators. In a preferred fashion the antiviral agent concerns protease inhibitors and/or reverse transcriptase inhibitors. The immunostimulators are preferably bropirimine, anti-human alpha-interferon antibodies, IL-2, GM-CSF, interferons, diethyl dithiocarbamate, tumor necrosis factors, naltrexone, tuscarasol and/or rEPO.

In another preferred embodiment of the invention the carriers are selected from the group comprising fillers, diluents, binders, humectants, disintegrants, dissolution retarders, absorption enhancers, wetting agents, adsorbents and/or lubricants.

The fillers and diluents are preferably starches, lactose, cane-sugar, glucose, mannitol and silica, the binder is preferably carboxymethylcellulose, alginate, gelatin, polyvinylpyrrolidone, the humectant is preferably glycerol, the disintegrant is preferably agar, calcium carbonate and sodium carbonate, the dissolution retarder is preferably paraffin, and the absorption enhancer is preferably a quaternary ammonium compound, the wetting agent is preferably cetyl alcohol and glycerol monostearate, the adsorbent is preferably kaolin and bentonite, and the lubricant is preferably talc, calcium and magnesium stearates and solid polyethylene glycols, or mixtures of the materials mentioned above.

The invention also relates to vectors, cells and/or organisms having a nucleoside of the invention, a nucleic acid of the invention and/or a pharmaceutical agent of the invention.

The invention also relates to the use of the nucleosides of the invention, the nucleic acids of the invention and/or the pharmaceutical agent of the invention in the prophylaxis or therapy of a viral, bacterial, fungicidal and/or parasitic infection or of cancer. For example, it is well-known to those skilled in the art that viruses can induce various tumors. Using the compounds of the invention, such tumors can be prevented prophylactically or treated therapeutically. Obviously, the structures of the invention can also be utilized in an anticancer combination therapy, for example. Those skilled in the art are also familiar with the fact that, in addition to viruses, bacteria associated with viral diseases or appearing by themselves represent a medical problem. Numerous bacteria have resistance to the well-known antibacterial agents. The compounds of the invention can be used in the prophylaxis and treatment of bacterial infections as well. Furthermore, the compounds of the invention can be used in the production of drugs for the treatment and prophylaxis of bacterial infections. In a preferred fashion the bacteria can be those from the genuses Escherichia coli, Salmonella spp., Shigella flexneri, Citrobacter freundii, Klebsiella pneumoniae, Vibrio spp., Haemophilus influenzae, Yersinia enterolitica, Pasturella haemolytica, and Proteus spp.

In another preferred embodiment the invention relates to the use of the compounds of the invention to prevent incorporation of other nucleosides during transcription in a growing DNA chain, prevent formation of a DNA-RNA hybrid, separate a base pair, or in competitive inhibition of a growing DNA chain.

In another preferred embodiment of the invention, the compounds of the invention are used in a prophylactic or therapeutic treatment of viral diseases associated with one of the following viruses or a combination thereof: hepatitis virus, HIV, bovine immunodeficiency virus, human T cell leukemia virus, feline immunodeficiency virus, caprine arthritis-encephalitis virus, equine infectious anemia virus, ovine Maedi-Visna virus, Visna-Lenti virus and others. In a preferred fashion, DNA viruses are treated. Those skilled in the art are familiar with the fact that the incidence of such viral infections can be combined with bacterial, fungicidal, parasitic or other infections.

Such use is particularly preferred in those cases where the hepatitis virus is a hepatitis B or a hepatitis D virus.

In a likewise particularly preferred fashion the pharmaceutical agent of the invention comprises inhibitors of HBV DNA polymerase. Obviously, the pharmaceutical agent for treatment, especially of hepatitis B, may include further effective anti-HBV agents, preferably PMEA (adefovir-dipivoxil), famciclovir, penciclovir, diaminopurine-dioxolane (DAPD), clevudine (L-FMAU), entecavir, interferon or thymosin α1 and/or inhibitors of nucleocapsid formation, particularly heteroarylpyrimidines.

In a likewise preferred fashion the agents are pegylated.

Moreover, it is particularly preferred that the agent includes additional agents capable of eliminating the function of cellular proteins essential to HBV growth.

In a likewise particularly preferred fashion, the above agent includes agents against viruses resistant to lamivudine or other cytosine nucleosides, such as emtricitabine (L-FTC), L-ddC, L-ddeC, L-dC and/or elvucitabine (L-fD4C). In a preferred fashion the agent can also be employed against liver carcinoma diseases triggered by chronic hepatitis, particularly by HBV.

In a likewise preferred fashion the β-L-nucleosides enhance the effect of other pharmaceutical agents in a non-additive, additive or synergistic fashion, increase the therapeutic index and/or reduce the risk of toxicity inherent in the respective compounds.

A preferred HIV in the meaning of the invention is HIV-1 with the subtypes A to J (HIV-1 group M) in accordance with the prior art subtype classification and the distantly related HIV-0 (HIV-1 group 0). Preferred main subtypes are 1A, 1B, 1C and 1D. The subtypes 1E, 1G and 1H are closely related to HIV-1A and likewise preferred. The preferred HIV-1A and 1C, as well as 1B and 1D show homology with respect to each other. The likewise preferred HIV-0 is more heterogeneous than HIV-1 in particular virus isolates. Classification into subtypes is not possible. Also preferred is HIV-2 which can be classified into the subtypes A to E. It has milder pathogenicity compared to HIV-1 and has therefore spread more slowly. The genetic variability results in changes in the external coat proteins. The influence on cytotropism, as well as the question to what extent this is accompanied by varying transmission probabilities have not been clarified sufficiently. Likewise preferred is treatment of double infections with different subtypes (e.g. B and E).

In a preferred embodiment of the invention the nucleosides of the invention are used in combination with 3-deazauridine. Combined use may involve simultaneous or time-shifted administration. Such combined administration can be effected in a combined agent, for example.

For example, the combined agent in the meaning of the invention can be such in nature that nucleosides of the invention and 3-deazauridine are included together in a solution or solid, e.g. in a tablet. In this event, the ratio of nucleosides of the invention and 3-deazauridine may vary freely. A ratio of nucleosides of the invention and 3-deazauridine ranging from 1:10,000 to 10,000:1 is preferred. The ratio of nucleosides of the invention and 3-deazauridine may vary within this range, depending on the desired application. Of course, said at least two components—nucleosides of the invention and 3-deazauridine—can also be incorporated together in a solution or solid in such a way that release thereof will proceed in a time-shifted fashion. However, the combined agent in the meaning of the invention may also be constituted of two separate solutions or two separate solids, one solution or solid essentially comprising 3-deazauridine and the other solution or solid essentially comprising the nucleosides of the invention. The two solutions or solids can be associated with a common carrier or with separate carriers. For example, the two solutions and/or the two solids can be present in a capsule as common carrier. Such a formulation of the combined agent of the invention is advantageous in those cases where administration of the nucleosides of the invention and 3-deazauridine is to proceed in a time-shifted manner. That is, the organism is initially contacted with nucleosides of the invention, e.g. by infusion or oral administration, to be contacted with the other component of the combined agent in a time-shifted manner. Of course, it is also possible to provide the combined agent by means of conventional pharmaceutical-technical methods and procedures in such a way that the organism is initially contacted with 3-deazauridine and subsequently with the nucleosides of the invention. Hence, the organism is contacted sequentially with the components of the combined agent. The time period between administration of the two components of the combined agent of the invention or the initial release of nucleosides of the invention or 3-deazauridine depends on the age, sex, overall constitution of the patient, the disease, or other parameters which can be determined by the attending physician using prior tests, for example.

In a particularly preferred embodiment of the invention the compounds of the invention are used as a prodrug, as feed additive and/or as drinking water additive, the use as feed additive and/or drinking water additive being preferred in veterinary medicine.

In a particularly preferred fashion the compounds of the invention are used as prodrug. The utilization of endocytosis for the cellular uptake of active substances comprising polar compounds is highly effective for some, particularly long-lived substances, but is very difficult to transfer to more general uses. One alternative is the prodrug concept generally known to those skilled in the art. By definition, a prodrug includes its active substance in the form of a non-active precursor metabolite. It is possible to distinguish between carrier prodrug systems, some of them being multi-component ones, and biotransformation systems. The latter include the active substance in a form requiring chemical or biological metabolization. Such prodrug systems are well-known to those skilled in the art, e.g. valacyclovir as a precursor of acyclovir, or others. Carrier prodrug systems include the active substance as such, bound to a masking group which can be cleaved off by a preferably simple controllable mechanism. The inventive function of masking groups in the nucleosides of the invention is neutralization of the negative charge on the phosphate residue for improved reception by cells. When using the nucleosides of the invention together with a masking group, the latter may also influence other pharmacological parameters, such as oral bioavailability, distribution in tissue, pharmacokinetics, as well as stability to non-specific phosphatases. In addition, delayed release of the active substance may entail a depot effect. Furthermore, modified metabolization may occur, thereby achieving higher efficiency of the active substance or organ specificity. In the event of a prodrug formulation, the masking group, or a linker group binding the masking group to the active substance, is selected in such a way that the nucleoside prodrug has sufficient hydrophilicity to be dissolved in the blood serum, sufficient chemical and enzymatic stability to reach the site of action, and hydrophilicity suitable for diffusion-controlled membrane transport. Furthermore, it should permit chemical or enzymatic liberation of the active sub-stance within a reasonable period of time and, of course, the liberated auxiliary components should not be toxic. In the meaning of the invention, however, the nucleoside with no mask or no linker and no mask can also be understood as prodrug because the structure inhibiting viral DNA polymerase is a high-energy triphosphate which initially must be provided via enzymatic and biochemical processes from the incorporated nucleoside in the cell.

In another particularly preferred embodiment of the invention the compounds of the invention are formulated as a gel, powder, tablet, sustained-release tablet, premix, emulsion, brew-up formulation, drops, concentrate, granulate, syrup, pellet, bolus, capsule, aerosoa, spray and/or inhalant and/or used in this form. The tablets, coated tablets, capsules, pills and granulates can be provided with conventional coatings and envelopes optionally including opacification agents, and can be composed such that release of the active substance(s) takes place only or preferably in a particular area of the intestinal tract, optionally in a delayed fashion, to which end polymer substances and waxes can be used as embedding materials.

Preferably, the drugs of the present invention can be used in oral administration in any orally tolerable dosage form, including capsules, tablets and aqueous suspensions and solutions, without being restricted thereto. In case of tablets for oral application, carriers frequently used include lactose and corn starch. Typically, lubricants such as magnesium stearate can be added. For oral administration in the form of capsules, diluents that can be used include lactose and dried corn starch. In oral administration of aqueous suspensions the active substance is combined with emulsifiers and suspending agents. Also, particular sweeteners and/or flavors and/or coloring agents can be added, if desired.

The active substance(s) can also be present in micro-encapsulated form, optionally with one or more of the above-specified carrier materials.

In addition to the active substance(s), suppositories may include conventional water-soluble or water-insoluble carriers such as polyethylene glycols, fats, e.g. cocoa fat and higher esters (for example, C₁₄ alcohols with C₁₆ fatty acids) or mixtures of these substances.

In addition to the active substance(s), ointments, pastes, creams and gels may include conventional carriers such as animal and vegetable fats, waxes, paraffins, starch, tragacanth, cellulose derivatives, polyethylene glycols, silicones, bentonites, silica, talc and zinc oxide or mixtures of these substances.

In addition to the active substance(s), powders and sprays may include conventional carriers such as lactose, talc, silica, aluminum hydroxide, calcium silicate and polyamide powder or mixtures of these substances. In addition, sprays may include conventional propellants such as chlorofluorohydrocarbons.

In addition to the active substance(s), solutions and emulsions may include conventional carriers such as solvents, solubilizers, and emulsifiers such as water, ethyl alcohol, isopropyl alcohol, ethyl carbonate, ethyl acetate, benzyl alcohol, benzyl benzoate, propylene glycol, 1,3-butylene glycol, dimethylformamide, oils, especially cotton seed oil, peanut oil, corn oil, olive oil, castor oil and sesame oil, glycerol, glycerol formal, tetrahydrofurfuryl alcohol, polyethylene glycols, and fatty esters of sorbitan, or mixtures of these substances. For parenteral application, the solutions and emulsions may also be present in a sterile and blood-isotonic form.

In addition to the active substance(s), suspensions may include conventional carriers such as liquid diluents, e.g. water, ethyl alcohol, propylene glycol, suspending agents, e.g. ethoxylated isostearyl alcohols, polyoxyethylenes orbitol and sorbitan esters, microcrystalline cellulose, aluminum metahydroxide, bentonite, agar, and tragacanth, or mixtures of these substances.

The drugs can be present in the form of a sterile injectable formulation, e.g. as a sterile injectable aqueous or oily suspension. Such a suspension can also be formulated by means of methods known in the art, using suitable dispersing or wetting agents (such as Tween 80) and suspending agents. The sterile injectable formulation can also be a sterile injectable solution or suspension in a non-toxic, parenterally tolerable diluent or solvent, e-g. a solution in 1,3-butanediol. Tolerable vehicles and solvents that can be used include mannitol, water, Ringer's solution, and isotonic sodium chloride solution. Furthermore, sterile, non-volatile oils are conventionally used as solvents or suspending medium. Any mild non-volatile oil, including synthetic mono- or diglycerides, can be used for this purpose. Fatty acids such as oleic acid and glyceride derivatives thereof can be used in the production of injection agents, e.g. natural pharmaceutically tolerable oils such as olive oil or castor oil, especially in their polyoxyethylated forms. Such oil solutions or suspensions may also include a long-chain alcohol, such as Ph.Helv., or a similar alcohol as diluent or dispersant.

The above-mentioned formulation forms may also include colorants, preservatives, as well as odor- and taste-improving additives, e.g. peppermint oil and eucalyptus oil, and sweeteners, e.g. saccharine. Preferably, the active sub-stances of formula (I) and (II), i.e., the nucleosides of the invention, should be present in the above-mentioned pharmaceutical preparations at a concentration of about 0.1 to 99.5 wt.-%, more preferably about 0.5 to 95 wt.-% of the overall mixture.

In addition to the compounds of formula (I) and (II), the above-mentioned pharmaceutical preparations may include further pharmaceutical active substances. The production of the pharmaceutical preparations specified above proceeds in a usual manner according to well-known methods, e.g. by mixing the active substance(s) with the carrier material(s).

The above-mentioned preparations can be applied in humans and animals on an oral, rectal, parenteral (intravenous, intramuscular, subcutaneous), intracisternal, intravaginal, intraperitoneal route, locally (powders, ointment, drops) and used in the therapy of infections in hollow areas and body cavities. Injection solutions, solutions and suspensions for oral therapy, gels, brew-up formulations, emulsions, ointments or drops are possible as suitable preparations. For local therapy, ophthalmic and dermatological formulations, silver and other salts, ear drops, eye ointments, powders or solutions can be used. With animals, ingestion can be effected via feed or drinking water in suitable formulations. Furthermore, gels, powders, tablets, sustained-release tablets, premixes, concentrates, granulates, pellets, boli, capsules, aerosols, sprays, inhalants can be used in humans and animals. Moreover, the compounds of the invention can be incorporated in other carrier materials such as plastics (plastic chains for local therapy) collagen or bone cement.

In another preferred embodiment of the invention the compounds of the invention, i.e., the nucleosides of the invention, the nucleic acids of the invention, the inventive pharmaceutical agents or vectors, cells and organisms, are incorporated in a preparation at a concentration of 0.1 to 99.5, preferably 0.5 to 95, and more preferably 20 to 80 wt.-%. That is, the compounds of the invention are pre-sent in the above-specified pharmaceutical formulations, e.g. tablets, pills, granulates and others, at a concentration of preferably 0.1 to 99.5 wt.-% of the overall mixture. Those skilled in the art will be aware of the fact that the amount of active substance, i.e., the amount of an inventive compound combined with the carrier materials to produce a single dosage form, will vary depending on the host to be treated and on the particular type of administration. Once the condition of a host or patient has improved, the proportion of active compound in the preparation can be modified so as to obtain a maintenance dose. Depending on the symptoms, the dose or frequency of administration or both can subsequently be reduced to a level where the improved condition is retained. Once the symptoms have been alleviated to the desired level, the treatment should be terminated. However, patients may require an intermittent treatment on a long-term basis if any symptoms of the disease should recur. Accordingly, the proportion of the compounds, i.e. their concentration, in the overall mixture of the pharmaceutical preparation, as well as the composition or combination thereof, is variable and can be modified and adapted by a person of specialized knowledge in the art.

Those skilled in the art will be aware of the fact that the compounds of the invention can be contacted with an organism, preferably a human or an animal, on various routes. Furthermore, a person skilled in the art will also be familiar with the fact that the pharmaceutical agents in particular can be applied at varying dosages. Application should be effected in such a way that a viral disease is combatted as effectively as possible or the onset of such a disease is prevented by a prophylactic administration. Concentration and type of application can be determined by a person skilled in the art using routine tests. Preferred applications of the compounds of the invention are oral application in the form of powders, tablets, juice, drops, capsules or the like, rectal application in the form of suppositories, solutions and the like, parenteral application in the form of injections, infusions and solutions, inhalation of vapors, aerosols and dusts and pads, and local application in the form of ointments, pads, dressings, ravages and the like. Contacting with the compounds according to the invention is preferably effected in a prophylactic or therapeutic fashion. In prophylactic administration, an infection with the above-mentioned viruses is to be pre-vented at least in such a way that, following invasion of single viruses, e.g. into a wound, further growth thereof is massively reduced or viruses having invaded are destroyed virtually completely. In therapeutic contacting, a manifest infection of the patient is already existing, and the viruses already present in the body are either to be destroyed or inhibited in their growth. Other forms of application preferred for this purpose are e.g. subcutaneous, sublingual, intravenous, intramuscular, intraperitoneal and/or topical ones.

For example, the suitability of the selected form of application, of the dose, application regimen, selection of adjuvant and the like can be determined by taking serum aliquots from the patient, i.e., human or animal, and testing for the presence of viruses, i.e., determining the virus titer, in the course of the treatment procedure. Alternatively or concomitantly, the condition of the liver, but also, the amount of T cells or other cells of the immune system can be determined in a conventional manner so as to obtain a general survey on the immunological constitution of the patient and, in particular, the constitution of organs important to the metabolism, particularly of the liver. Additionally, the clinical condition of the patient can be observed for the desired effect, especially the anti-infectious, preferably antiviral effect. As set forth above, especially hepatitis, but also HIV or other diseases can be associated with other e.g. bacterial or fungicidal infections or tumor diseases, for which reason additional clinical co-monitoring of the course of such concomitant infections or tumor diseases is also possible. Where insufficient therapeutic effectiveness is achieved, the patient can be subjected to further treatment using the agents of the invention, optionally modified with other well-known medicaments expected to bring about an improvement of the overall constitution. Obviously, it is also possible to modify the carriers or vehicles of the pharmaceutical agent or to vary the route of administration. In addition to oral ingestion, e.g. intramuscular or subcutaneous injections or injections into the blood vessels can be envisaged as another preferred route of therapeutic administration of the compounds according to the invention. At the same time, supply via catheters or surgical tubes can also be used. In addition to the above-specified concentrations during use of the compounds of the invention, the compounds in a preferred embodiment can be employed in a total amount of 0.05 to 500 mg/kg body weight per 24 hours, preferably 5 to 100 mg/kg body weight. Advantageously, this is a therapeutical quantity which is used to prevent or improve the symptoms of a disorder or of a responsive, pathologically physiological condition. The amount administered is sufficient to prevent or inhibit infection or spreading of an infectious agent such as hepatitis B or HIV in the recipient. The effect of the compounds of the invention on the above-mentioned viruses, with respect to their prophylactic or therapeutic potential, is seen e.g. as an inhibition of the viral infection, inhibition of syncytium formation, inhibition of fusion between virus and target membrane, as a reduction or stabilization of the viral growth rate in an organism, or in another way. For example, the therapeutic effect can be such that, as a desirable side effect, particular antiviral medicaments are improved in their effect or, by reducing the dose, the number of side effects of these medicaments will be reduced as a result of applying the compounds of the invention. Of course, the therapeutic effect also encompasses direct action on the viruses in a host. That is, however, the effect of the compounds of the invention is not restricted to eliminating the viruses, but rather comprises the entire spectrum of advantageous effects in prophylaxis and therapy. Obviously, the dose will depend on the age, health and weight of the recipient, degree of the disease, type of required simultaneous treatment, frequency of the treatment and type of the desired effects and side-effects. The daily dose of 0.05 to 500 mg/kg body weight can be applied as a single dose or multiple doses in order to furnish the desired results. The dose levels per day can be used in prevention and treatment of a viral infection, including hepatitis B infection. In particular, pharmaceutical agents are typically used in about 1 to 7 administrations per day, or alternatively or additionally as a continuous infusion. Such administrations can be applied as a chronic or acute therapy. Of course, the amounts of active substance that are combined with the carrier materials to produce a single dosage form may vary depending on the host to be treated and on the particular type of administration. In a preferred fashion, the daily dose is distributed over 2 to 5 applications, with 1 to 2 tablets including an active substance content of 0.05 to 500 mg/kg body weight being administered in each application. Of course, it is also possible to select a higher content of active substance, e.g. up to a concentration of 5000 mg/kg. The tablets can also be sustained-release tablets, in which case the number of applications per day is reduced to 1 to 3. The active substance content of sustained-release tablets can be from 3 to 3000 mg. If the active substance—as set forth above—is administered by injection, the host is preferably contacted 1 to 8 times per day with the compounds of the invention or by using continuous infusion, in which case quantities of from 1 to 4000 mg per day are preferred. The preferred total amounts per day were found advantageous both in human and veterinary medicine. It may become necessary to deviate from the above-mentioned dosages, and this depends on the nature and body weight of the host to be treated, the type and severity of the disease, the type of formulation and application of the drug, and on the time period or interval during which the administration takes place. Thus, it may be preferred in some cases to contact the organism with less than the amounts mentioned above, while in other cases the amount of active substance specified above has to be surpassed. A person of specialized knowledge in the art can determine the optimum dosages required in each case and the type of application of the active substances.

In another particularly preferred embodiment of the invention the compounds of the invention, i.e., the nucleoside, the nucleic acid, the pharmaceutical agent, the vector, the cells and/or organism, are used in a single administration of from 1 to 80, especially from 3 to 30 mg/kg body weight. In the same way as the total amount per day, the amount of a single dose per application can be varied by a person of specialized knowledge in the art. Similarly, the compounds used according to the invention can be employed in veterinary medicine with the above-mentioned single concentrations and formulations together with the feed or feed formulations or drinking water. A single dose preferably includes that amount of active substance which is administered in one application and which normally corresponds to one whole, one half daily dose or one third or one quarter of a daily dose. Accordingly, the dosage units may preferably include 1, 2, 3 or 4 or more single doses or 0.5, 0.3 or 0.25 single doses. In a preferred fashion, the daily dose of the compounds according to the invention is distributed over 2 to 10 applications, preferably 2 to 7, and more preferably 3 to 5 applications. Of course, continuous infusion of the agents according to the invention is also possible.

In a particularly preferred embodiment of the invention, 1 to 2 tablets are administered in each oral application of the compounds of the invention. The tablets according to the invention can be provided with coatings and envelopes well-known to those skilled in the art or can be composed in a way so as to release the active substance (s) only in preferred, particular regions of the host.

In another preferred embodiment of the invention the compounds according to the invention can be employed together with at least one other well-known pharmaceutical agent. That is to say, the compounds of the invention can be used in a prophylactic or therapeutic combination in connection with well-known drugs. Such combinations can be administered together, e.g. in an integrated pharmaceutical formulation, or separately, e.g. in the form of a combination of tablets, injection or other medications administered simultaneously or at different times, with the aim of achieving the desired prophylactic or therapeutic effect. These well-known agents can be agents which enhance the effect of the nucleosides according to the invention. In the antibacterial sector, in particular, it was found that a wide variety of antibiotics improve the effect of nucleosides. This includes agents such as benzylpyrimidines, pyrimidines, sulfoamides, rifampicin, tobramycin, fusidinic acid, clindamycin, chloramphenicol and erythromycin. Accordingly, another embodiment of the invention relates to a combination wherein the second agent is least one of the above-mentioned antiviral or antibacterial agents or classes of agents. It should also be noted that the compounds of tile invention and combinations can also be used in connection with immune-modulating treatments and therapies.

Typically, there is an optimum ratio of compound(s) of the invention with respect to each other and/or with respect to other therapeutic or effect-enhancing agents (such as transport inhibitors, metabolic inhibitors, inhibitors of renal excretion or glucuronidation, such as probenecid, acetaminophen, aspirin, lorazepan, cimetidine, ranitidine, colifibrate, indomethacin, ketoprofen, naproxen etc.) where the active substances are present at an optimum ratio. Optimum ratio is defined as the ratio of compound(s) of the invention to other therapeutic agent(s) where the overall therapeutic effect is greater than the sum of the effects of the individual therapeutic agents. In general, the optimum ratio is found when the agents are present at a ratio of from 10:1 to 1:10, from 20:1 to 1:20, from 100:1 to 1:100 and from 500:1 to 1:500. In some cases, an exceedingly small amount of a therapeutic agent will be sufficient to increase the effect of one or more other agents. In addition, the use of the compounds of the invention in combinations is particularly beneficial in order to reduce the risk of developing resistance. Of course, the compounds of the invention, such as nucleosides or nucleic acids, can be used in combination with other well-known antiviral agents. Such agents are well-known to those skilled in the art. Accordingly, the compounds of the invention can be administered together with all conventional agents, especially other drugs, available for use particularly in connection with hepatitis drugs, either as a single drug or in a combination of drugs. They can be administered alone or in combination with same.

In a preferred fashion the compounds of the invention are administered together with said other well-known pharmaceutical agents at a ratio of about 0.005 to 1. Preferably, the compounds of the invention are administered particularly together with virus-inhibiting agents at a ratio of from 0.05 to about 0.5 parts to about 1 part of said known agents. In this event, tumor-inhibiting or antibacterial agents can be concerned. The pharmaceutical composition can be present in substance or as an aqueous solution together with other materials such as preservatives, buffer substances, agents to adjust the osmolarity of the solution, and so forth.

The invention also relates to the use of the nucleic acids of the invention as antisense nucleic acids, particularly in an antiviral therapy. Those skilled in the art are familiar with the fact that nucleic acids can be used as anti-sense nucleic acids. In a preferred fashion the nucleic acid of the invention serves to prevent hybridization of the RNA during translation, and this proceeds via hybridization of the viral RNA with the nucleic acids according to the invention. More specifically, the nucleic acids of the invention can be used as agents against hepatitis B because degradation thereof by cellular restriction enzymes is absent or difficult. In general, the nucleic acid of the invention hybridizes with the DNA of the hepatitis B virus, thereby not only impeding translation, but also transcription into viral DNA.

The nucleosides and nucleic acids according to the invention can be used in the production of pharmaceutical agents. Thus, the teaching of the invention may also relate to a method for the treatment of a viral, bacterial, fungicidal and/or parasitic infection or of cancer, in which method the nucleosides and/or nucleic acids of the invention are contacted with an organism. Treatment in the meaning of the invention includes both prophylactic and therapeutic treatment. In a preferred fashion the compounds of the invention can be used to protect organisms, especially human patients, from viral infection during a particular incident, such as delivery, or for a prolonged period of time, in a country where high risk of hepatitis B infection exists. In such cases, the compounds of the invention can be used alone or together with other prophylactic agents or other antiviral agents enhancing the efficacy of the respective agent. Preferably following oral application, the nucleosides of the invention advantageously can undergo easy absorption into the bloodstream of mammals, especially human mammals. Advantageously, the compounds exhibit good water solubility and consistent oral availability. In particular, it is said good oral availability that makes the compounds of the invention excellent agents for orally administered cures of treatment and prevention against viral infection, especially hepatitis B infection. Of course, the compounds of the invention not only are orally bioavailable, but advantageously have also a high therapeutic index which, in particular, is a measure of toxicity versus anti-viral effect. Accordingly, the compounds of the invention are more effective at lower dose levels compared to selected well-known antiviral agents, avoiding the toxic effect associated with these medical substances. The potential of the compounds of the invention of being released at doses far exceeding their active antiviral range is particularly advantageous in slowing down or preventing possible development of resistant variants. During a prophylactic treatment, in particular, the compounds of the invention can be used in a healthy, but also in a virally infected, especially in a hepatitis B virus infected patient, either as a single agent or together with other antiviral agents preferably impairing the replication cycle of hepatitis viruses. The use of the compounds of the invention in prophylaxis and therapy proceeds in a way well-known to those skilled in the art. In those cases where the method of treating a viral infection with the nucleosides of the invention represents a combination therapy, each agent used, i.e., both the well-known compounds and the compounds of the invention, has an additive, non-additive or synergistic effect in inhibiting virus replication, because action of each agent at a different site of replication of the viruses advantageously can be envisaged. Advantageously, the method of such combination therapies can also reduce the dosage of a conventional antiviral agent which, in comparison (when administering the agent alone), would be required for a desired therapeutic or prophylactic effect. Such combinations in the method of the invention for the treatment of viral diseases can reduce or eliminate the side effects of conventional therapies using single antiviral agents, and such combinations advantageously do not impair but rather synergistically increase the antiviral effect of these agents. These combinations reduce the potential of resistance to therapy using single agents, while advantageously minimizing the toxicity associated therewith. These combinations can also increase the efficacy of conventional agents without increasing the toxicity associated therewith. In a particularly preferred fashion the compound according to this invention, together with other antiviral or antibacterial or fungicidal agents, prevent replication of the genetic material of viruses in an additive or synergistic manner. Inter alia, preferred combination therapies include the administration of a compound of the invention together with ddC, d4T, 3TC or a combination thereof. Of course, administration together with other nucleoside derivatives or viral reverse transcriptase inhibitors or protease inhibitors may also be preferred in the method of the invention or in the use according to the invention. Joint administration of the compounds of the invention and viral reverse transcriptase inhibitors or aspartyl protease inhibitors shows an additive or synergistic effect, thereby preventing, essentially reducing or completely eliminating virus replication or infection or both, or symptoms associated therewith. Administration of a combination of agents can be preferred over administration of single agents. The compounds of the invention can also be used together with immunomodulators or immunostimulators; preferred immunomodulators or immunostimulators are: bropirimine, anti-human α-interferon antibodies, IL-2, GK-CSF, interferon α, diethyl dithiocarbamate, tumor necrosis factor, naltrexone, tuscarasol, rEPO and antibiotics such as pentamidine isethioriate, but also agents preventing or combatting malignant tumors associated with viral diseases. In the method for the treatment of viral, bacterial, fungicidal and/or parasitic infections or of cancer, the compounds of the invention—as set forth above—can be administered together with tolerable carriers, adjuvants or vehicles. Pharmaceutically tolerable carriers, adjuvants and vehicles that can be used in the drugs of this invention include ion exchangers, aluminum oxide, aluminum stearate, lecithin, self-emulsifying drug delivery systems (SEDDS), such as d-α-tocopherol-polyethylene glycol 1000 succinate, or other similar polymer delivery matrices, serum proteins such as human serum albumin, buffer substances such as phosphates, glycine, sorbic acids, potassium sorbate, partial glyceride mixtures of saturated vegetable fatty acids, water, salts or electrolytes such as protamin sulfate, disodium hydrogen phosphate, potassium hydrogen phosphate, sodium chloride, zinc salts, colloidal silicon dioxide, magnesium trisilicates, polyvinylpyrrolidone, materials on cellulose basis, polyethylene glycol, sodium carboxymethylcellulose, polyacrylates, waxes, polyethylene-polyoxypropylene block polymers, polyethylene glycol, and wool fat, but are not restricted thereto. Cyclodextrins such as α-, β-, and γ-cyclodextrin or chemically modified derivatives such as hydroxyalkylcyclodextrins, including 2- and 3-hydroxypropyl-β-cyclodextrins or other solubilized derivatives, can also be used with advantage in order to enhance delivery of the compounds according to the invention. In the context with this method, the compounds of the invention can be administered orally, parenterally, via inhalation spray, topically, rectally, nasally, buccally, vaginally, or via implanted reservoirs. Oral administration or administration via injection is a preferred form of contacting. The drugs of this invention may include any conventional non-toxic, pharmaceutically tolerable carriers, adjuvants or vehicles. In some cases, the pH value of the formulation can be adjusted using pharmaceutically tolerable acids, bases or buffers in order to increase the stability of the formulated compound or delivery form thereof. The term parenteral, as used herein, includes subcutaneous, intracutaneous, intravenous, intramuscular, intra-articular, intrasynovial, intrasternal, intrathecal, intralesional and intracranial injection or infusion procedures as a form of contacting.

The invention also relates to a kit comprising the compounds of the invention, optionally together with information on how to combine the contents of the kit. The information for combining the contents of the kit relates to the use of said kit in the prophylaxis and/or therapy of diseases, particularly viral diseases. For example, the information may also concern a therapeutic scheme, i.e., a concrete injection or application scheme, the dose to be administered, or other.

The nucleoside analogs of the invention have many advantages. In the course of their individual development, human and animal organisms must cope with numerous pathogens. For example, these pathogens can be fungi, bacteria, but also viruses, in particular. Each year, millions of people and economically useful animals develop a viral disease, and a large number of such infections are accompanied by significant health impairments. Untreated for a prolonged period of time, diseases with human immunodeficiency virus and hepatitis viruses can be fatal.

The viruses an organism has to cope with strongly differ in their infectious potential. Highly infectious viruses include hepatitis B virus (HBV) which may cause inflammations of the liver, regularly accompanied by liver cell damage, and such liver damage can develop up to a liver tumor in chronic courses with selected viruses, such as hepatitis viruses B, C and D.

To allow successful combatting of viruses in a host organism, e.g. in a human or in a farm or domestic animal, the prior art has developed various antiviral therapies. A large number of these therapies are chemotherapies intended to prevent replication of pathogenic viruses in a host cell. Various phases of replication, such as adsorption, penetration, translation, transcription of the viral genes, replication of nucleic acids, as well as assembly of virus particles, are possible as targets of attack for the so-called virustatic agents used to this end. Virus adsorption inhibitors interact with cationic regions of the viral coat protein, thereby preventing association with receptors of the potential host cell. In contrast to the adsorption inhibitors, the inhibitors of virus cell fusion do not act as early as to prevent binding, but rather act at a later stage to prevent fusion with the host cell to form a common membrane. Another way would be inhibition of penetration with liberation of the viral genome, as has been described in the prior art, e.g. for Picorna viruses. Furthermore, it is possible to block the transcription and protein biosynthesis of viruses. Methods of inhibiting viral DNA polymerase have also been described in the prior art. The inhibition of viral DNA polymerase has been disclosed in the prior art particularly for herpes viruses. The DNA polymerase of herpes viruses assumes various functions. Among other things, it is responsible for the introduction of the viral genetic information into the host cell genome, for RNA-dependent DNA synthesis, for DNA-dependent DNA synthesis, and has additional functions. A large number of presently known, successfully applied antiviral compounds are nucleoside-analogous substances which, however, are limited in their antiviral activity to herpes viruses in particular.

As the above-mentioned strategies are successful in herpes viruses, in particular, and allow application to other viruses with less success in some cases, it has been necessary to develop different therapies for each particular group of viruses. Thus, for example, vaccines produced by genetic engineering have been available for years for the treatment of hepatitis B; however, they fail to be helpful in individuals already infected and exert significant influence on the above-mentioned chronic course of said disease. The nucleosides of the invention avoid the above-specified drawbacks of the prior art.

Without intending to be limiting, the invention will be explained in more detail with reference to the following examples.

### EXAMPLES

### 1. Synthesis of 4-hydroxyaminopyrimidin-2(1H)-one β-L-Nucleosides from the Corresponding Uracil or Thymine Nucleosides

### 1.1 Synthesis of 1-(2-deoxy-β-L-ribofuranosyl)-4-hydroxyaminopyrimidin-2(1H)-one (β-L-N4-hydroxydeoxycytidine)

1-(2,3-Di-O-benzoyl-2-deoxy-β-L-ribofuranosyl)uracil (1.3 g, 2.98 mmol) was dissolved in triethylamine (1.8 ml, 12.9 mmol) and anhydrous acetonitrile (70 ml). The solution was cooled to 0° C. in an argon atmosphere and mixed with 2,4,6-triisopropylbenzenesulfonyl chloride (1.95 g, 6.3 mmol) and 4-dimethylaminopyridine (300 mg, 2 mmol). The reaction mixture was left at room temperature overnight with stirring. Subsequently, hydroxylamine hydrochloride (450 mg, 6.47 mmol) was added and the reaction solution was stirred at room temperature for 24 hours. Thereafter, water (50 ml) and chloroform (75 ml) were added. The organic phase was washed with saturated sodium chloride solution and dried over sodium sulfate. The residue obtained after removing the solvent in vacuum was purified by means of column chromatography on silica gel, using chloroform/methanol (98/2, v/v) as eluent. 1-(2,3-Di-O-benzoyl-β-L-ribofuranosyl)-4-hydroxyamino-pyrimidin-2(1H)-one was isolated from the corresponding fractions as a white amorphous mass (1.7 g).

The above amount of substance was added to ammonia-saturated methanol (20 ml). The reaction solution was left for 24 hours at room temperature and was subsequently concentrated to dryness in vacuum. The residue was purified by means of column chromatography on silica gel, using a chloroform/methanol (9/1, v/v) mobile phase. 1-(2-Deoxy-β-L-ribofuranosyl)-4-hydroxyaminopyrimidin-2(1H)-one was obtained from the corresponding fractions and crystallized from methanol/ether (yield: 232 mg, 0.94 mmol, 31, 6%).

### 1.2 Synthesis of 1-(2-Deoxy-β-L-ribofuranosyl)-4-hydroxyamino-5-methylpyrimidin-2(1H)-one (β-L-5-methyl-N4-hydroxydeoxycytidine)

According to the general synthetic method described above and starting from 1-(3,5-di-O-acetyl-2-deoxy-β-L-ribofuranosyl)thymine (500 mg, 1.53 mmol), β-L-5-methyl-N4-hydroxydeoxycytidine was obtained (132 mg, 0.5 mmol, 32%).

### 1.3 Synthesis of 1-(2-deoxy-β-L-ribofuranosyl) 5-fluoro-4-hydroxyaminopyrimidin-2(1H)-one (β-L-5-fluoro-N4-hydroxydeoxycytidine)

β-L-5-Fluoro-2′-deoxyuridine was prepared according to established methods for the synthesis of the corresponding D-derivative (Ozaki et al., Bull Chem Soc Japan 1977, 50: 2197-2198).

A stirred solution of 1-(5-O-acetyl-2-deoxy-β-L-ribofuranosyl)-5-fluorouracil (288 mg, 1 mmol) in anhydrous acetonitrile (30 ml) under an argon atmosphere was cooled to 0° C. To this solution were successively added 2,4,6-triisopropyl benzenesulphonyl chloride (654 mg, 2.1 mmol) and 4-dimethylaminopyridine (132 mg, 1 mmol). The resulting mixture was stirred for 20 h at room temperature. Solid hydroxylamine hydrochloride (149 mg, 2.1 mmol) was added and the mixture was stirred for an additional 24 h. The mixture was partitioned between water (25 ml) and chloroform (100 ml). The organic layer was washed with a saturated aqueous sodium chloride solution (30 ml), dried over anhydrous sodium sulfate, filtered, and the solvent was removed under reduced pressure. The resulting residue was purified by column chromatography on silica gel eluting with a gradient of methanol (0-10%) in chloroform to afford 1-(5-O-acetyl-2-deoxy-β-L-ribofuranosyl)-5-fluoro-4-hydroxyaminopyrimidin-2(1H)-one as a white solid (138 mg, 0.45 mmol). A solution of this compound in methanol saturated with ammonia at 0° C. was kept for 24 h at room temperature. After removing of the solvent under reduced pressure the residue was purified by column chromatography on silica gel with chloroform/methanol (9/1, v/v) as eluent to afford 1-(2-deoxy-β-L-ribofuranosyl)-5-fluoro-4-hydroxyaminopyrimidin-2(1H)-one (94 mg, 036 mmol) as a white solid.

### 1.4 Synthesis of 1-(2,3-dideoxy-β-L-glycero-pentofuranosyl)-5-fluoro-4-hydroxyaminopyrimidin-2(1H)-one (β-L-2′, 3′-dideoxy-5-fluoro-N4-hydroxycytidine)

β-L-2′,3′-Didehydro-2,3′-dideoxy-5-fluorouridine was pre-pared according to established methods described for the synthesis of the corresponding D-derivative (Joshi et al., J Chem Soc Perkin Trans I 1992, 2537-2544). This compound was acetylated in the usual manner with acetanhydride in pyridine and purified by column chromatography. The isolated product was activated with 2,4,6-triisopropyl benzenesulphonyl chloride and 4-dimethylaminopyridine, then reacted with solid hydroxylamine hydrochloride as described in example 1.3. The reaction product was purified by column chromatography to afford the acetylated N4 hydroxycytidine derivative.

A solution of 1-(5-O-acetyl-2,3-dideoxy-β-L-glycero-pent-2-enofuranosyl)-5-fluoro-4-hydroxyaminopyrimidin-2(1H)-one (285 mg, 1 mmol) in dioxane was catalytically hydrogenolyzed as described in example 1.6.

The product of that reaction was deacetylated by treatment with a solution of ammonia in methanol (saturated at 0° C.) for 24 h. The solvent was removed under reduced pressure. The residue was purified by column chromatography on silica gel eluting with chloroform/methanol (95/5, v/v). 1-(2,3-dideoxy-β-L-glycero-pentofuranosyl)-5-fluoro-4-hydroxyaminopyrimidin-2(1H)-one was afforded as a white foam (67 mg, 0.27 mmol).

¹H-NMR (DMSO-d₆) δ 10.43, 9.99 (2H, s, NH-4, OH-4), 7.54 (1H, d, H-6), 5.73 (1H, t, H-1′), 5.21 (1H, t, OH-5′), 4.23-4.18 (1H, m, H-4′), 3.70-3.45 (2H, m, H-5′, H-5″), 2.17-2.04 (4H, m, H-3′, H-3″, H-2′, H-2′).

### 1.5 Synthesis of 1-(2,3-dideoxy-β-L-glycero-pent-2-enofuranosyl)-4-hydroxyaminopyrimidin-2(1H)-one(β-L-2′,3′-didehydro-2′,3′-dideoxy-N4-hydroxycytidine)

β-L-2′,3′-Didehydro-2′,3′-dideoxyuridine was prepared according to established methods described for the synthesis of the corresponding D-derivative (Horwitz et al., J Org Chem 1966, 31:205-211).

1-(5-O-Acetyl-2,3-dideoxy-β-L-glycero-pent-2-enofuranosyl)-uracil (288 mg, 1 mmol) was dissolved in dioxane (30 ml) cooled to 0° C. To this solution were added successively under an argon atmosphere 2,4,6-triisopropylbenzenesulphonyl chloride (654 mg, 2.1 mmol) and 4-dimethylaminopyridine (132 mg, 1 mmol).

This solution was stirred for 24 h at room temperature. Hydroxylamine hydrochloride (149 mg, 2.1 mmol) was then added, and the mixture was further stirred fox 1 day at room temperature. Water (25 ml) was added, and the product was extracted with chloroform (100 ml). The organic layer was washed with a aqueous solution saturated with sodium chloride (30 ml), dried over anhydrous sodium sulfate, filtered, and concentrated under reduced pressure.

The resulting residue was purified by column chromatography on silica gel eluting with a gradient of methanol (0-5%) in chloroform to give 1-(5-O-acetyl-2,3-dideoxy-β-L-glycero-pent-2-enofuranosyl)-4-hydroxyaminopyrimidin-2(1H)-one as a white foam. This compound was concentrated vacuo, the residue was purified by column chromatography on silica gel eluting with chloroform/methanol (95/5, v/v) to afford 1-(2,3-dideoxy-β-L-glycero-pent-2-enofuranosyl)-4-hydroxyaminopyrimidin-2(1H)-one (79 mg, 0.35 mmol).

¹H-NMR (DMSO-d₆) δ 10.96, 10.01 (2H, 2s, NH-4, OH-4), 7.64 (1H, s, H-6), 6.58 (1H, d, H-1′), 6.37 (1H, dd, H-3′), 6.23 (1H, dd, H2′), 5.53 (1H, d, H-5), 5.21 (1H, t, OH-5′), 4.20-4.12 (1H, m, H-4′), 3.75-3.52 (2H, m, H-5′, H-5″).

### 1.6 Synthesis of 1-(2,3-dideoxy-β-L-glycero-pentofuranosyl)-4-hydroxyaminopyrimidin-2(1H)-one (β-L-2′,3′-dideoxy-N4-hydroxycytidine)

β-L-2′,3′-Didehydro-2′,3′-dideoxyuridine was prepared according to established methods described for the synthesis of the corresponding D-derivative (Horwitz et al., J Org Chem 1966, 31:205-211).

This deoxyuridine derivative was acetylated with acetanhydride in pyridine. The reaction product was purified by column chromatography. The isolated derivative was activated with 2,4,6-triisopropyl benzenesulphonyl chloride and 4-dimethylaminopyridine in acetonitrile, then hydroxylamino chloride was added and the reaction mixture was worked up as described in example 1.3. After evaporation of the solvent the acetylated hydroxycytidine derivative was purified by column chromatography.

A solution of 1-(5-O-acetyl-2,3-dideoxy-β-L-glycero-pent-2-enofuranosyl)-4-hydroxyaminopyrimidin-2(1H)-one (267 mg, 1 mmol) in dioxane containing 125 mg of 10% palladium-charcoal catalyst was shaken with 1 atm. of hydrogen at room temperature. The theoretical uptake of hydrogen was realized in 0.5 h, the catalyst was filtered, and the filtrate was evaporated to dryness.

The residue was treated with methanol/ammonia (25 ml) overnight at room temperature. After removing the solvent the corresponding residue was purified by column chromatography on silica gel with chloroform/methanol (9/1, v/v) as solvent to afford 1-(2,3-dideoxy-β-L-glycero-pentofuranosyl)-4-hydroxyaminopyrimidin-2(1H)-one (105 mg, 0.46 mmol) as a solid.

¹H-NMR (DMSO-d₆) δ 10.41, 9.95 (2H, 2s, NH-4, OH-4), 7.54 (1H, d, H-6), 5.73 (1H, d, H-5), 5.58 (1H, t, H-1′), 5.03 (1H, t, OH-5′), 4.94 (m, 1H, H-4′), 3.51 (m, 2H, H-5′, H-5), 2.31-2.56 (m, 4H, H-3′, H-3″, H-2′, H-2″).

### 1.7 Synthesis of 1-(2,3-dideoxy-β-L-glycero-pent-2-eno-furanosyl)-5-fluoro-4-hydroxyaminopyrimidin-2(1H)-one (β-L-2′,3′-Didehydro-2′,3′-dideoxy-5-fluoro-N4-hydroxycytidine)

β-L-2′,3′-Didehydro-2′,3′-dideoxy-5-fluorouridine was prepared according established methods for synthesis of the corresponding D-derivative (Joshi et al., J Chem Soc Perkin Trans I 1992, 2537-2544).

In a similar manner as described under example 15 using 1-(5-O-acetyl-2,3-dideoxy-β-L-glycero-pent-2-enofuranosyl)-5-fluorouracil (252 mg, 1 mmol) as starting material, the title compound 1-(2,3-dideoxy-β-L-glycero-pent-2-enofuranosyl)-5-fluoro-4-hydroxyaminopyrimidin-2(1H)-one was obtained (69 mg, 0.33 mmol).

### 1.8 Synthesis of 1-(2,3-dideoxy-β-L-glycero-pent-2-enofuranosyl)-5-methyl-4-hydroxyaminopyrimidin-2(1H)-one

(β-L-2′,3′-didehydro-2′,3′-dideoxy-5-methyl-N4-hydroxycytidine)

2′,3′-Didehydro-2′,3′-deoxy-β-L-thymidine (5-L-thymidinene) was prepared according to established methods described for synthesis of corresponding D-derivative (Horwitz et al., J Org Chem 1966, 31: 205-211). β-L-thymidinene was acetylated in the usual manner with acetanhydride in pyridine. 5′-O-acetyl-2′,3′-didehydro-2′, 3′-deoxy-β-L-thymidine (266 mg, 1 mmol) was subjected to the same sequence of reaction steps as described in the example 1.5 to afford 1-(2,3-dideoxy-5-L-glycero-pent-2-enofuranosyl)-5-methyl-4-hydroxy-aminopyrimidin-2(1H)-one (132 mg, 0.55 mmol).

¹H-NMR (DMSO-d₆) δ10.44, 10.02 (2H, s, NH-4, OH-4), 7.63 (1H, s, H-6), 6.81 (1H, dd, H-1′), 6.42 (1H, m, H-3′), 5.95 (1H, m, H-2′), 5.02 (1H, brt, OH-5′), 4.78 (1H, m, H-4′), 3.62 (2H, m, H-5′, H-5″), 1.78 (3H, s, CH₃).

### 2. Synthesis of 4-hydroxyaminopyrimidin-2(1H)-one β-L-Nucleosides from the Corresponding Cytosine Nucleosides

### 2.1 Synthesis of S-L-2′,3′-dideoxy-3′-thia-N4-hydroxycytidine

β-L-2′,3′-Dideoxy-3′-thiacytidine was synthesized as described (Beach et al., J Org Chem 1992, 57: 2217-2219). 500 mg (2.18 mmol) of it was mixed with a 7 M hydroxylamine hydrochloride solution (25 ml). The reaction solution was kept at room temperature for four days with stirring. Following removal of the solvent in vacuum, the resulting residue was purified by means of column chromatography on silica gel, using the upper phase of the mixture ethyl acetate/isopropanol/water (4/1/2, v/v/v) as eluent.

The solvent of the corresponding fractions was removed in vacuum. β-L-2′,3′-dideoxy-3′-thia-N4-hydroxycytidine was obtained from the methanol solution of the residue (yield: 95 mg, 0.39 mmol, 17.9%).

### 2.2 Synthesis of 1-(2,3-dideoxy-2-fluoro-β-L-glycero-pent-2-enofuranosyl)-4-hydroxyaminopyridin-2(1H)-one(β-L-2′,3′-didehydro-2,3′-dideoxy-2′-fluoro-N4-hydroxycytidine)

β-L-2′,3′-Didehydro-2′,3′-dideoxy-2′-fluorocytidine was synthesized as described (Lee et al., J Med Chem 1999, 42:1320-1328).

400 mg (1.76 mmol) of this compound was dissolved in 10 ml of 5 M hydroxylamine hydrochloride which had been adjusted to pH 6.0 with sodium hydroxide. The solution was stirred for 24 h at room temperature, and the solvent was removed under reduced pressure.

The residue was purified by column chromatography on silica gel with chloroform/methanol (9/1, v/v) as eluent to afford 1-(2,3-dideoxy-2-fluoro-β-L-glycero-pent-2-enofuranosyl)-4-hydroxyaminopyridin-2(1H)one (83 mg, 0.34 mmol, yield 19.3%).

### 2.3 Synthesis of β-L-[2-(hydroxymethyl)-1,3-oxathiolan-4-yl]-5-fluoro-4-hydroxyaminopyridin-2(1H)-one(β-L-2′,3′-dideoxy-3′-thia-5-fluoro-N4-hydroxycytidine)

β-L-2,3′-Dideoxy-3′-thia-5-fluorocytidine was synthesized as described (Beach et al., J Org Chem 1992, 57: 2217-2219).

78 mg (0.31 mmol) of this compound was shaken for 24 h in 2 ml of aqueous 5 M hydroxylamine hydrochloride (adjusted to pH 6.0).

The solvent was removed in vacuo, and the residue was purified by column chromatography on silica gel eluting with chloroform/methanol (9/1, v/v). From the corresponding fractions β-L-[2-(hydroxymethyl)-1,3-oxa-thiolan-4-yl]-5-fluoro-4-hydroxyaminopyridin-2(1H)-one was isolated as a foam (14 mg, 0.05 mmol, yield 16%).

### 2.4 Synthesis of 1-(3-azido-2,3-dideoxy-β-L-ribofuranosyl)-4-hydroxyaminopyridin-2(1H)-one (β-L-3′-azido-2′,3′-dideoxy-N4-hydroxycytidine)

β-L-3-Azido-2′,3′dideoxycytidine was prepared according to established methods described for the synthesis of the corresponding D-derivative. 300 mg, (1.2 mmol) of this compound was dissolved in 10 ml aqueous 5 M hydroxylamine hydrochloride (adjusted to pH 6.0) and treated according example 2.2. The title compound was obtained as a white solid (103 mg, 0.38 mmol, yield 31.6%).

### 2.5 Synthesis of 1-(3-azido-2,3-dideoxy-β-L-ribofuranosyl)-5-fluoro-4-hydroxyaminopyridin-2(1H)-one (β-L-3′-azido-2′,3′-dideoxy-5-fluoro-N4-hydroxycytidine)

β-L-3′-Azido-2′,3′dideoxy-5-fluorocytidine was prepared according to established methods described for the synthesis of the corresponding D-derivative (Sandström et al., Drugs 1986, 31: 462-467).

500 mg (1.85 mmol) of this compound were treated as described in example 2.2. The title compound β-L-3′-azido-2′,3′-dideoxy-5-fluoro-N4-hydroxycytidine (121 mg, 0.42 mmol, yield 22.7%) was obtained.

### 2.6 Synthesis of 1-(3-azido-2,3-dideoxy-β-L-ribofuranosyl)-5-methyl-4-hydroxyaminopyridin-2(1H)-one (β-L-3-azido-2′,3′-dideoxy-5-methyl-N4-hydroxycytidine)

β-L-3′-Azido-2′, 3′dideoxy-5-methylcytidine was prepared according to established methods described for the synthesis of the corresponding D-derivative (Lin et al., J Med Chem 1983, 26: 544-551).

450 mg, (1.69 mmol) of this compound were treated as described in example 2.2. The title compound β-L-3′-azido-2′,3′-dideoxy-5-methyl-N4-hydroxycytidine (143 mg, 0.5 mmol, yield 29.5%) was obtained.

### 2.7 Synthesis of 1-(2,3-dideoxy-3-fluoro-β-L-ribofuranosyl)-4-hydroxyaminopyridin-2(1H)-one (β-L-2′,3′-dideoxy-3′-fluoro-N4-hydroxycytidine)

β-L-2′,3′-Dideoxy-3′-fluorocytidine was synthesized as described (von Janta-Lipinski et al. J Med Chem 1998, 12: 2040-2046.)

This compound (350 mg, 1.52 mmol) gave according to the synthetic method described in example 2.2, 1-(2,3-dideoxy-3-fluoro-β-L-ribofuranosyl)-4-hydroxyaminopyridin-2(1H)-one (137 mg, 0.56 mmol, yield 36.8%) as a solid.

¹H-NMR (DMSO-d₆) δ 10.48, 10.06 (2H, s, NH-4, OH-4), 7.76 (1H, d, H-6), 6.25 (1H, m, H-1′), 5.47 (1H, d, H-5), 5.25 (1H, dd, H-3′, J_{F-3}=53.6 Hz), 5.11 (1H, t, OH-5′), 4.13 (1H, dt, H-4′, J_{F-4}=27 Hz), 3.52-3.64 (2H, m, H-5′, H-5″), 2.38-2.45 (2H, m, H-2′, H-2″).

### 3. Inhibition of HBV-Replication by the Compounds of Invention in HepG2 2.2.15 Cells

The antiviral efficacy of the compounds of the invention was investigated on HepG2 2.2.15 cells, a human hepatoblastoma cell line which has the replication-competent HBV genome stably integrated therein and produces infectious progeny viruses in a productive manner (Sells et al., Proc Natl Acad Sci USA 1987, 84: 1005-1009).

The above cells were cultured under standardized conditions as specified by Korba and Gerin, and the amount of extracellular viral DNA was determined (Korba et al., Antiviral Res 1992, 19: 55-70).

Following passaging, the HepG2 2.2.15 cells were seeded at a density of about 60% in 12-well plates and cultured to confluence in 10% FBS Dulbecco MEM. Thereafter, the medium was changed to 2% FBS, and the cells were cultured for another 24 hours.

After another change of medium, the cells were treated with varying concentrations of compounds according to the invention. Every 24 hours the compounds were re-added together with the medium. On the 6^{th} day of treatment, the cell supernatants were centrifuged off and stored at −20° C. until analysis of the HBV DNA was effected.

Following treatment of the culture supernatants with proteinase K, the extracellular viral DNA was amplified by means of PCR using the following primers (forward: 5′-CTC CAG TTC AGG AAC AGT AAA CCC-3′; reverse: 5′-TTG TGA GCT CAG AAA GGC CTT GTA AGT TGG CG-31. The PCR products were separated on 1% agarose, stained with ethidium bromide and quantified using a Fluor-S™ Multimager (Biorad).

For calibration of the PCR reaction, serial dilutions of the pUC19 HBV and pTHBV plasmids with known genome equivalents (GE) were used, resulting in a lower detection limit of about 10³ GE and a linearity between 10³ and 10⁵ GE. Table 1 shows the concentrations of compounds of the invention required for 50% reduction of extracellular HBV DNA (ED₅₀) after 6 days incubation of the HepG2 2.2.15 cells.

Between the new compounds β-L-2′,3′-didehydro-2′,3′-dideoxy-N4-hydroxycytidine (L-HyddeC), 5-L-2′,3′-didehydro-2′,3′-dideoxy-5-fluoro-N4-hydroxycytidine (L-HyddeFC) and β-2′,3′-didehydro-2′,3′-dideoxy-2′-fluoro-N4-hydroxycytidine (L-HyFddeC) were the most effective nucleoside with EC₅₀-values of <0.1 μM.

A second group of compounds including β-L-2′-3′-dideoxy-3′-thia-N4-hydroxycytidine (Hy3TC), β-L-2′-3′-dideoxy-3′-thia-5-fluoro-N4-hydroxycytidine (HyFTC), β-L-2′,3′-dideoxy-N4-hydroxycytidine (L-HyddC), and β-L-2′,3′-dideoxy-5-fluoro-N4-hydroxycytidine (L-HyddFC) gave EC₅₀-values between 0.3 and 0.65 μM.

A third group of compounds of the invention with EC₅₀-values between 3 and 50 μM includes β-L-N4-hydroxydeoxycytidine (L-HyCdR), β-L-5-fluoro-N4-hydroxydeoxycytidine (L-HyFCdR), β-L-5-methyl-N4-hydroxydeoxycytidine (L-HyMetCdR), β-L-3′-fluoro-2′,3′-dideoxy-N4-hydroxycytidine (L-FHyCdR), and β-L-3′-azido-2′, 3′-dideoxy-N4-hydroxycytidine (L-N₃HyCdR).

It can be argued that the N-4-hydroxy-group of the β-L-cytidine derivatives could be metabolized inside of cells to the corresponding NH₂-group. Such a reaction could suggest a prodrug function of the presented analogues. In this case Hy3TC as the prodrug of 3TC should also display a high efficiency against HIV because 3TC inhibits the HIV-replication at a EC₅₀ of 0.002 μM (Schinazi et al., Antimicrob Agents Chemother 1992, 38: 2423-2431).

However, we found that Hy3TC is inactive against HIV replication (EC₅₀>>25 μM) ruling out the possibility that the metabolic conversion of the NHOH-group to the NH₂-group could be the reason for its anti-HBV activity.

TABLE 1
Inhibition of HBV-replication in HepG2 2.2.15 cells by β-L-
hydroxycytosine nucleosides compared to 3TC(lamivudine), β-L-
dideoxycytidine(L-ddC), β-L-thymidine(L-TdR), β-L-5-fluorode-
oxycytidine(L-FCdR). The concentrations required for 50% reduction
of HBV DNA in the medium of the cells are given(EC₅₀;
μM).

| Compound | EC₅₀; μM |
|---|---|
| 3TC (lamivudine) | 0.1 |
| L-ddC | 0.25 |
| L-TdR | 0.3 |
| L-FCdR | 1.2 |
| L-HyCdR | 3.0 |
| L-HyFCdR | 4.5 |
| L-HyMetCdR | 7.8 |
| L-FHyCdR | 25 |
| L-N₃HyCdR | 50 |
| Hy3TC | 0.5 |
| HyFTC | 0.3 |
| L-HyddC | 0.65 |
| L-HyddFC | 0.35 |
| L-HyddeC | 0.05 |
| L-HyddeFC | <0.1 |
| L-HyFddeC | <0.1 |

Abbreviations:
3TC (lamivudine) = 2,3′-dideoxy-3′-thiacytidine;
L-HyCdR = β-L-N4-hydroxydeoxycytidine;
L-HyFCdR = β-L-5-fluoro-N4-hydroxydeoxycytidine;
L-HyMetCdR = β-L-5-methyl-N4-hydroxydeoxycytidine;
L-FHyCdR = β-L-3′-fluoro-2′,3′-dideoxy-N4-hydroxycytidine;
L-N₃HyCdR = β-L-3′-azido-2′,3′di-deoxy-N4-hydroxycytidine;
Hy3TC = β-L-2′-3′-dideoxy-3′-thia-N4-hydroxycytidine;
HyFTC = β-L-2′-3′-dideoxy-3′-thia-5-fluoro-N4-hydroxycytidine;
L-HyddC = β-L-2′,3′-dideoxy-N4-hydroxycytidine;
L-HyddFC = β-L-2′,3′-dideoxy-5-fluoro-N4-hydroxycytidine;
L-HyddeC = β-L-2′,3′-didehydro-2′,3′-dideoxy-N4-hydroxycytidine;
L-HyddeFC = β-L-2′,3′-didehydro-2′,3′-dideoxy-5-fluo-ro-N4-hydroxycytidine;
L-HyFddeC = β-L-2′,3′-didehydro-2′,3′-dideoxy-2′-fluoro-N4-hydroxycytidine.

### 4. Inhibition of HBV DNA Polymerase by β-L-N4-hydroxycytosine Nucleoside Triphosphates

Synthesis and purification of the triphosphates of β-L-N4-hydroxycytosine nucleosides were performed according to well-known methods (Yoshikawa et al., Tetrahedron Lett 1967, 50: 5065-5068; Hoard et Ott, J Am Chem Soc 1965, 87: 1785-1788).

To determine the endogenous HBV DNA polymerase activity, about 100 ml of serum from patients with untreated hepatitis B virus infections from Charité, Berlin, (>10⁷ HBV particles/ml), was centrifuged at 3000 rpm. Virus particles of the cleared serum were sedimented in a Beckman SW28 rotor at 25,000 rpm, 60 min. The virus pellet was suspended in 7 ml of TKM buffer (50 mM Tris-HCl, pH 7.5, 50 mM KCl, 5 mM MgCl₂), Layered over a step gradient of 10 ml each of 0.3 M, 0.6 M, 0.9 M saccharose in TKM buffer and centrifuged at 25,000 rpm for 20 hours.

The purified virus pellet was suspended in 250 μl TKM buffer, lysed by ultrasound, divided in aliquots and frozen at −80° C. (Davies et al., Antiviral Res 1996, 30: 133-145).

The HBV DNA polymerase assay contained in 30 μl about 2-4×10⁸ purified virus particles (lysed beforehand additionally in 6% β-mercaptoethanol, 10% Igepal for 15 min at room temperature), 42 mM Tris-HCl, (pH 7.5), 34 mM MgCl₂, 340 mM KCl, 22 mM β-mercaptoethanol, 0.4% Igepal, 70 μM TTP, dATP, dGTP and 1 μCi ³H-dCTP (=0.7 μM dCTP) (Matthes et al., Antimicrob Agents & Chemother 1991, 35: 1254-1257) and varying concentrations of β-L-N4-hydroxycytosine nucleoside triphosphates as inhibitors.

Following a one-hour incubation at 37° C., 20 μl of the assay volume was placed on paper filter, washed 5 times with 5% trichloroacetic acid and 0.1% Na pyrophosphate, and the ³H-dCMP incorporated in the HBV DNA was subsequently measured in a Liquid Scintillation Counter.

Using the concentration-dependent inhibition curves of HBV DNA synthesis, the concentration of 1-L-N4-hydroxycytosine nucleoside triphosphates resulting in 50% inhibition of the HBV DNA polymerase activity was determined.

Table 2 demonstrates that the HBV DNA polymerase is inhibited strongly by the triphosphates of L-Hy3TC, L-HyddC and L-HyddeC (IC₅₀ between 0.15 and 0.65 μM) pointing out that the 4-NHOH-group of the cytosine nucleoside triphosphates is effective at the target and does not require a previous metabolization to the NH₂-group.

TABLE 2
Inhibition of HBV DNA polymerase by triphosphates of β-L-
hydroxycytidine nucleoside analogues in comparison to 3TC-tri-
phosphate(IC₅₀).

| Triphosphate | |
|---|---|
| of | IC₅₀; μM |
| 3TC (lamivudine) | 0.30 |
| L-HyCdR | 6.0 |
| L-HyMetCdR | 4.0 |
| L-Hy3TC | 0.65 |
| L-HyddC | 0.55 |
| L-HyddeC | 0.28 |

### 5. Cytotoxicity of D-L-N4-hydroxycytosine Nucleosides

To this end, established cells of a human myeloid leukemia (HL-60) in RPMI medium, and the above-mentioned HepG2 cells in Dulbecco MEM, respectively, were incubated for two days using varying concentrations of compounds, and the proliferation rate of the cells was subsequently determined. The data were used to determine the concentration of compounds resulting in 50% inhibition of proliferation (CD₅₀). Table 3 shows that the new compounds display no antiproliferative activity on HepG2- and HL-60 cells.

Remarkably, also L-HyddC, L-HyddeFC and L-HyFddeC have lost the antiproliferative activity which was described for the corresponding cytosine analogues containing the q-NH₂-group instead of the 4-NHOH-group (IC₅₀ for L-ddC=70 μM, Lin et al., J Med Chem 1994, 37: 798-803; IC₅₀ for ddeFC=7 μM, Lin et al., J Med Chem 1996, 39: 1757-1759; IC₅₀ for L-FddeC=100 μM, Lee et al., J Med Chem 1999, 42: 1320-1328).

Thus these data further support our suggestion that the NH₂-group could not be formed inside of cells from our β-L-N4-hydroxycytosine nucleoside analogues.

TABLE 3
Cytotoxicity of β-L-N4-hydroxycytosine-nucleosides
against HepG2-and HL-60 cells in comparison to 3TC(lamivudine).
Concentrations producing 50% inhibition of cell proliferation
were given (CD₅₀).

CD₅₀; μM

| Compound | HepG2-cells | HL-60 cells |
|---|---|---|
| 3TC (lamivudine) | 1900 | 2000 |
| L-HyCdR | 545 | 460 |
| L-HyFCdR | 920 | 1370 |
| L-HyMetCdR | 490 | 600 |
| L-FhyCdR | 820 | 4400 |
| L-N₃HyCdR | 1160 | 2000 |
| Hy3TC | 1230 | 1450 |
| HyFTC | 975 | 1100 |
| L-HyddC | 2250 | 1600 |
| L-HyddFC | 1580 | 1860 |
| L-HyddeC | >5000 | >3500 |
| L-HyFddeC | 1370 | 1130 |

Abbreviations:
3TC (lamivudine) = 2,3′-dideoxy-3′-thiacytidine;
L-HyCdR = β-L-N4-hydroxydeoxycytidine;
L-HyFCdR = β-L-5-fluoro-N4-hydroxydeoxycytidine;
L-HyMetCdR = β-L-5-methyl-N4-hydroxydeoxycytidine;
L-FHyCdR = β-L-3′-fluoro-2′,3′-dideoxy-N4-hydroxycytidine;
L-N₃HyCdR = β-L-3′-azido-2′,3′-di-deoxy-N4-hydroxycytidine;
Hy3TC = β-L-2′-3′-dideoxy-3′-thia-N4-hydroxycytidine;
HyFTC = β-L-2′-3′-dideoxy-3′-thia-5-fluoro-N4-hydroxycytidine;
L-HyddC = β-L-2′,3′-dideoxy-N4-hydroxycytidine;
L-HyddFC = β-L-2′,3′-dideoxy-5-fluoro-N4-hydroxycytidine;
L-HyddeC = β-L-2′,3′-didehydro-2′,3′-dideoxy-N4-hydroxycytidine;
L-HyddeFC = β-L-2′,3′-didehydro-2′,3′-dideoxy-5-fluoro-N4-hydroxycytidine;
L-HyFddeC = β-L-2′,3′-didehydro-2′,3′-dideoxy-2′-fluoro-N4-hydroxycytidine.

The state of the art may disclose more common empirical formulae, which do not however describe the special, chosen chemical compounds of the doctrine according to the application. Those special, precise compounds of the invention had not yet been made accessible in the form of common terms and conceptions, since it was not possible to generate exactly the compounds of the invention only by conducting routine experiments; those compounds show surprising, unobvious characteristics, for example the fact that hitherto all efforts of experts in this matter were in vain, a different approach to the development of scientific technology, the achievement forwards the development, misconceptions about the solution of the according problem (prejudice), technical progress (such as: improvement, increased performance, price-reduction, saving of time, material, work steps, costs or resources that are difficult to obtain, improved reliability, remedy of defects, improved quality, increased efficiency, augmentation of technical or medical possibilities, provision of another product, spare product, alternatives, enrichment of the pharmaceutical fund), a special choice (since a certain possibility, the result of which was unforeseeable, was chosen among a great number of possibilities).

The precise, claimed chemical compounds of the application have not yet been disclosed in greater fields that are comprised by a common formula. The precisely chosen compounds of the invention are not arbitrarily chosen specimen, but it is rather the selective choice that leads to products with the above-mentioned surprising characteristics.

1. New β-L-N4-hydroxycytosine deoxynucleotides of general formula I for the treatment and prophylaxis of HBV and HIV infections 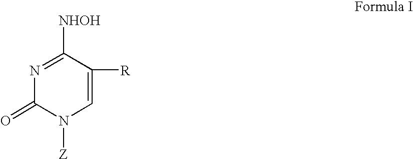
wherein:
R═H, halogen (F, Cl, Br, I), C₁-C₃ alkyl, and 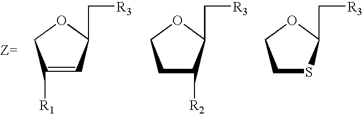
wherein
R₁═H, F;
R₂═H, F, OH, N₃; and
R₃═OH, O-acetyl, O-palmitoyl, alkoxycarbonyl, carbamate, phosphonate, monophosphate, bis(S-acyl-2-thioethyl) phosphate, diphosphate or triphosphate.

2. The β-L-nucleosides according to claim 1, wherein R═H, F, Cl, Br, I, or CH₃.

3. The β-L-nucleosides according to claim 1, wherein
R═H, F or CH₃, and
R₁═H or F, preferably H,
R₂═H, F, OH or N₃, and
R₃═OH.

4. A β-L-nucleoside according to claim 1, said β-L-nucleoside being
β-L-N4-hydroxydeoxycytidine,
β-L-5-methyl-N4-hydroxydeoxycytidine,
β-L-5-fluoro-N4-hydroxydeoxycytidine,
β-L-2′,3′-dideoxy-N4-hydroxycytidine,
β-L-2′,3′-dideoxy-5-fluoro-N4-hydroxycytidine,
β-L-2′,3′-didehydro-2′,3′-dideoxy-N4-hydroxycytidine,
β-L-2′,3′-didehydro-2′,3′-dideoxy-5-fluoro-N4-hydroxycytidine,
β-L-2′,3′-didehydro-2′,3′-dideoxy-5-methyl-N4-hydroxycytidine,
β-L-2′,3′-didehydro-2′,3′-dideoxy-2′-fluoro-N4-hydroxycytidine,
β-L-2′,3′-dideoxy-3′-thia-N4-hydroxycytidine,
β-L-2′,3′-dideoxy-3′-thia-5-fluoro-N4-hydroxycytidine,
β-L-3′-azido-2′,3′-dideoxy-N4-hydroxycytidine,
β-L-3′-azido-2′,3′-dideoxy-5-fluoro-N4-hydroxycytidine,
β-L-3′-azido-2′,3′-dideoxy-5-methyl-N4-hydroxycytidine, and
β-L-3′-fluoro-2′,3′-dideoxy-N4-hydroxycytidine.

5. The β-L-nucleoside according to claim 1, selected from the group consisting of a salt, a phosphonate, a monophosphate, bis(S-acyl-2-thioethyl) phosphate, diphosphate, triphosphate, an other ester and a salt of such ester.

6. The β-L-nucleoside according to claim 1 for producing a drug for the treatment and prophylaxis of HBV and HIV infections.

7. Immunostimulatory nucleic acids or oligonucleotides for treatment of cancer, HBV- and HIV-infections, asthma and allergic diseases containing a central de-oxycytidyl-deoxyguanosine dinucleotide (CpG) in which the deoxycytidine is replaced by β-L-N4-hydroxydeoxycytidine, β-L-5-methyl-N4-hydroxydeoxycytidine, or β-L-5-fluoro-N4-hydroxydeoxycytidine.

8. A pharmaceutical agent comprising a β-L-nucleoside or a derivative according to claim 1 and/or a nucleic acid containing a central de-oxycytidyl-deoxyguanosine dinucleotide (CpG) in which the deoxycytidine is replaced by β-L-N4-hydroxydeoxycytidine, β-L-5-methyl-N4-hydroxydeoxycytidine, or β-L-5-fluoro-N4-hydroxydeoxycytidine, optionally together with conventional auxiliaries, preferably carriers, adjuvants and/or vehicles.

9. The pharmaceutical agent according to claim 8, further comprising one or more additional agents from the group of antiviral, fungicidal or antibacterial agents, anti-cancer agents and/or immunostimulators or immunomodulators.

10. The pharmaceutical agent according to claim 9, wherein the antiviral agents are protease inhibitors and/or reverse transcriptase inhibitors and/or inhibitors of HBV DNA polymerase, the immunostimulators bropirimine, anti-human alpha-interferon antibodies, IL-2, GM-CSF, interferons, diethyl dithiocarbamate, tumor necrosis factors, naltrexone, tuscarasol and/or rEPO.

11. The pharmaceutical agent according to claim 8, comprising one or more additional anti-HBV-effective agents from the group comprising PMEA (adefovir-dipivoxil), famciclovir, penciclovir, diaminopurine-dioxolane (DAPD), clevudine (L-FMAU), telbivudine (L-Thymidine) entecavir, interferon or thymosin al and/or inhibitors of nucleocapsid formation, particularly heteroarylpyrimidines.

12. The pharmaceutical agent according to claim 8, wherein the agents are pegylated.

13. The pharmaceutical agent according to claim 8 further comprising one or more additional agents capable of eliminating the function of cellular proteins essential to HBV growth.

14. The pharmaceutical agent according to claim 8, wherein said agent is effective against hepatitis B viruses resistant to lamivudine or other cytosine nucleosides such as emtricitabine (L-FTC), L-ddC, L-ddeC, L-dC and/or elvucitabine (L-Fd4C).

15. The pharmaceutical agent according to claim 8, wherein said agent prevents cancer.

16. The pharmaceutical agent according to claim 8, wherein said agent prevents formation of liver carcinoma resulting from chronic hepatitis triggered by HBV.

17. The pharmaceutical agent according to claim 8, wherein the carriers are selected from the group comprising fillers, diluents, binders, humectants, disintegrants, dissolution retarders, absorption enhancers, wetting agents, adsorbents and/or lubricants.

18. A method for prophylaxis or therapy of a viral, bacterial, fungicidal and/or parasitic infection, or of cancer comprising:
administering to a person in need of such prophylaxis or therapy the β-L-nucleosides of claim 1 in a viral, bacterial, fungicidal and/or parasitic infection, or cancer prophylaxis or therapy effective amount.

19. The method of claim 18, wherein the viral infection is associated with hepatitis virus, HIV, bovine immunodeficiency virus, caprine arthritis-encephalitis virus, equine infectious anemia virus, ovine Maedi-Visna virus, Visna-Lenti virus, avian leukosis virus, human T cell leukemia virus, and/or feline immunodeficiency virus.

20. The method of claim 19, wherein the hepatitis virus is a hepatitis B or hepatitis D virus.

21. The method of claim 19, wherein the HIV is HIV-0, HIV-1 and/or HIV-2.

22. The β-L-nucleoside according to claim 1, wherein said β-L-nucleoside is a prodrug, a feed additive and/or a drinking water additive.

23. The method of claim 1, wherein the said β-L-nucleoside is in form of a gel, poudrage, powder, tablet, sustained-release tablet, premix, emulsion, brew-up formulation, drops, concentrate, granulate, syrup, pellet, bolus, capsule, aerosol, spray and/or inhalant.

24. The β-L-nucleoside according to claim 1, wherein said β-L-nucleoside is present in a preparation at a concentration of from 0.1 to 99.5, preferably from 0.5 to 95, more preferably from 20 to 80 wt.-%.

25. The method of claim 18, wherein said β-L-nucleoside is administered orally, rectally, subcutaneously, intravenously, intramuscularly, intraperitoneally and/or topically mute.

26. The method of claim 18, wherein said β-L-nucleoside is administered in overall amounts of from 0.05 to 500 mg/kg, preferably from 1 to 100 mg/kg body weight per 24 hours.

27. The method of claim 18, wherein the β-L-nucleoside and/or a nucleic acid containing a central de-oxycytidyl-deoxyguanosine dinucleotide (CpG) in which the deoxycytidine is replaced by β-L-N4-hydroxydeoxycytidine, β-L-5-methyl-N4-hydroxydeoxycytidine, or β-L-5-fluoro-N4-hydroxydeoxycytidine are employed administered in a single administration of from 1 to 80, preferably from 3 to 30 mg/kg body weight.

28. The method of claim 18, wherein said β-L-nucleoside is administered distributed over 2 to 10, preferably 3 to 5 daily applications.

29. The method of claim 25, wherein 1 to 2 tablets are administered in each oral application.

30. The method of claim 18, wherein said β-L-nucleoside is used in combination with at least one other well-known pharmaceutical agent.

31. The method of claim 30, wherein said β-L-nucleoside enhances the therapeutic effect of said other pharmaceutical agents in a non-additive, additive or synergistic fashion, increase the therapeutic index and/or reduce the risk of toxicity inherent in the respective compound.

32. The method of claim 30, wherein said β-L-nucleoside is administered together with said other well-known pharmaceutical agents at a ratio of about 0.005 to 1.

33. The method of claim 30, wherein said at least one β-L-nucleoside is used in combination with 3-deazauridine.

34. (canceled)

35. (canceled)

36. A kit comprising (a) the β-L-nucleoside according to claim 1 and/or (b) a nucleic acid containing a central de-oxycytidyl-deoxyguanosine dinucleotide (CpG) in which the deoxycytidine is replaced by β-L-N4-hydroxydeoxycytidine, β-L-5-methyl-N4-hydroxydeoxycytidine, or β-L-5-fluoro-N4-hydroxydeoxycytidine and/or the pharmaceutical agent comprising (a) and/or (b) optionally comprising a β-L-nucleoside or a derivative according to claim 1 and/or a nucleic acid containing a central de-oxycytidyl-deoxyguanosine dinucleotide (CpG) in which the deoxycytidine is replaced by β-L-N4-hydroxydeoxycytidine, β-L-5-methyl-N4-hydroxydeoxycytidine, or β-L-5-fluoro-N4-hydroxydeoxycytidine, optionally together with conventional auxiliaries, Preferably carriers, adjuvants and/or vehicles, optionally together as well as with information for combining the contents of the kit.

37. Use of the kit according to claim 36 prophylaxis or therapy of viral diseases.

38. A pharmaceutical combination preparation comprising at least one β-L-nucleoside according to claim 1 and 3-deazauridine for the treatment and/or prophylaxis of HBV and HIV infections.
